(19) United States
(12) Patent Application Publication
(10) Pub. No.: US 2024/0192195 A1
(43) Pub. Date: Jun. 13, 2024

(54) A MULTIOMIC APPROACH TO MODELING OF GENE REGULATORY NETWORKS IN MULTIPLE MYELOMA

(71) Applicant: H. LEE MOFFITT CANCER CENTER AND RESEARCH INSTITUTE, INC., Tampa, FL (US)

(72) Inventors: Ariosto S. SILVA, Tampa, FL (US); Praneeth SUDALAGUNTA, Tampa, FL (US); Kenneth SHAIN, St. Petersburg, FL (US); Rafael Renatino CANEVAROLO, Tampa, FL (US); Mark B. MEADS, Tampa, FL (US)

(21) Appl. No.: 18/554,500

(22) PCT Filed: Apr. 11, 2022
(86) PCT No.: PCT/US2022/024217

§ 371 Date: Oct. 9, 2023

Related U.S. Application Data
(60) Provisional application No. 63/173,389, filed on Apr. 10, 2021.
(60) Provisional application No. 63/178,193, filed on Apr. 22, 2021.
(60) Provisional application No. 63/301,507, filed on Jan. 21, 2022.

(51) Int. Cl. G01N 33/50; C12Q 1/6886; G16B 20/00; G16B 40/20; G16H 10/20; G16H 20/10; G16H 30/40

(52) U.S. Cl. CPC G01N 33/5011; C12Q 1/6886; G16B 20/00; G16B 40/20; G16H 10/20; G16H 20/10; G16H 30/40; C12Q 2600/106; C12Q 2600/158

(57) ABSTRACT

Disclosed are methods for identifying a gene regulatory network and treatment regimens combined with MM standard of care drugs to either delay, or reverse resistance to the standard of care therapy. Also disclosed is a synergy between Selinexor (SELI) and dexamethasone (DEX), pomalidomide (POM), elotuzumab (ELO), and daratumumab (DARA), and expression signatures and mutations associated with response to these agents.

### CROSS-REFERENCE TO RELATED APPLICATIONS

This application claims the benefit of U.S. provisional patent application No. 63/173,389, filed on Apr. 10, 2021, and titled “SELINEXOR SYNERGISM IN MULTIPLE MYELOMA;” U.S. provisional patent application No. 63/178,193, filed on Apr. 22, 2021, and titled “MULTIOMIC APPROACH TO MATHEMATICAL MODELING OF GENE REGULATORY NETWORKS IN MULTIPLE MYELOMA;” and U.S. provisional patent application No. 63/301,507, filed on Jan. 21, 2022, and titled “MULTIOMIC APPROACH TO MATHEMATICAL MODELING OF GENE REGULATORY NETWORKS IN MULTIPLE MYELOMA,” the disclosures of which are expressly incorporated herein by reference in their entireties.

### STATEMENT REGARDING FEDERALLY FUNDED RESEARCH

This invention was made with government support under Grant No. U54CA193489 awarded by the National Cancer Institute, National Institutes of Health. The government has certain rights in the invention.

### I. BACKGROUND

Evolution of therapy resistance is a key challenge in cancer that leads to poor patient survival and unnecessary drug toxicity. Drug resistance is of particular interest in incurable cancers, where prolonging a patient's lifespan requires evading therapy resistance. Multiple myeloma (MM) is one such incurable but treatable bone marrow-resident plasma cell malignancy. Despite a number of effective anti-MM agents, often administered in the form of combinations of 3-4 drugs, patients' responses to successive lines of therapy are invariably followed by relapses, with increasingly short-lived intervening responses, ultimately leading to patient death due to evolution of multi-drug resistant disease. More importantly, there are no biomarkers for choice of therapy in MM. Thus, choice of therapy in heavily treated patients relies on clinical acumen and general guidelines. New Methods are needed for assessing patient responsiveness to therapeutics and therapeutic regiments.

### II. SUMMARY

Disclosed herein are methods of measuring tumor chemosensitivity in a subject with multiple myeloma comprising obtaining multiple myeloma cells from a subject; culturing said multiple myeloma cells; contacting the multiple myeloma cells with one or more individual anti-cancer agents and/or combinations of two or more anti-cancer agents; taking an image (such as, for example a bright field image) of said multiple myeloma cells at least two times; and applying an image analysis algorithm to said images to determine viability (including, but not limited to assessing non-translational cellular membrane motion (i.e., undulations of the cell membrane that are not caused by a cell moving from point a to point b, but a change in cell membrane motion and/or integrity) of the cell after stage drift and field vibrations are excluded) across time and/or concentration thereby forming a model of drug sensitivity.

In one aspect, disclosed herein are methods of measuring tumor chemosensitivity of any preceding aspect, wherein the multiple myeloma cells are cultured in the presence of stroma, collagen, and/or plasma.

Also disclosed herein are methods of measuring tumor chemosensitivity of any preceding aspect, wherein at least one image is obtained prior to the contact with the anti-cancer agent

In one aspect, disclosed herein are methods of measuring tumor chemosensitivity of any preceding aspect, wherein the multiple myeloma cells are imaged for at least 2, 3, 4, 5, 6, 7, 8, 9, 10 11, 12, 13, 14, 15, 16, 17, 18, 19, 20, 21, 22, 23, 24, 25, 26, 27, 28, 29, 30, or 31 days and/or wherein an image is made of the multiple myeloma cells every 5, 10, 15, 20, 25, 30, 35, 40, 45, 50, 55, 60, 65, 70, 75, 80, 85, 90, 95, 100, 105, 110, 115, 120 min, 3, 4, 5, 6, 7, 8, 9, 10, 11, 12, 13, 14, 15, 16, 17, 18, 19, 20, 21, 22, 23, 24, 36, 48, 60, 72 hours.

Also disclosed herein are methods of measuring tumor chemosensitivity of any preceding aspect, wherein the multiple myeloma cells are separately contacted with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more individual anti-cancer agents and/or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, or more combinations of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 individual anti-cancer agents.

In one aspect, disclosed herein are methods of measuring tumor chemosensitivity of any preceding aspect, further comprising coupling drug sensitivity model to clinical trials to establish a patient's response to single agents and combinations thereby establishing a patient's early objective response (EOR) to each of the drugs tested and quantify synergistic effects in combinations.

Also disclosed herein are methods of measuring tumor chemosensitivity of any preceding aspect, further comprising applying or adjusting a patient's treatment regimen based on the sensitivities.

In one aspect, disclosed herein are methods of identifying gene signatures associated with anti-cancer therapy resistance for multiple myeloma comprising obtaining multiple myeloma cells from a subject; culturing said multiple myeloma cells; contacting the multiple myeloma cells with one or more individual anti-cancer agents and/or combinations of two or more anti-cancer agents; taking an image of said multiple myeloma cells at least two times; applying an image analysis algorithm to said images to determine viability across time and/or concentration thereby forming a model of drug sensitivity; in parallel to assaying cellular sensitivity to one or more anti-cancer agents, sequencing multiple myeloma cells obtained from the patient; analyzing the gene expression profile obtained from the sequence information in combination with the drug sensitivity data to determine gene signatures associated with therapy resistance.

Also disclosed herein are methods of identifying gene signatures associated with anti-cancer therapy resistance of any preceding aspect, further comprising applying gene signature data to a gene regulatory network (GRN) model to identify transcriptional regulatory mechanisms driving therapy resistance.

In one aspect, also disclosed herein are methods of identifying gene signatures associated with anti-cancer therapy resistance of any preceding aspect, further comprising repeating the analysis from two or more patients to identify gene signatures and/or transcriptional regulatory mechanisms driving therapy resistance common to a cohort.

Also disclosed herein are methods of identifying therapeutic regimens for a subject comprising identifying gene signatures using the method of any preceding aspect and identifying transcriptional regulatory mechanisms driving therapy resistance using the method of any preceding aspect; applying gene signature and GRN model information to identify novel therapeutic strategies either as a combination, or sequential therapy.

In one aspect, disclosed herein are methods of identifying novel therapeutic regimens for the treatment of multiple myeloma comprising identifying gene signatures using the method of any preceding aspect from two or more patients and identifying transcriptional regulatory mechanisms driving therapy resistance using the method of any preceding aspect from two or more patients; applying gene signature and GRN model information to identify novel therapeutic strategies either as a combination, or sequential therapy.

Disclosed herein is a synergy between Selinexor (SELI) and dexamethasone (DEX), pomalidomide (POM), elotuzumab (ELO), and daratumumab (DARA), and expression signatures and mutations associated with response to these agents.

Also disclosed herein is a framework that relies on ex vivo drug response and RNA sequencing data for a cohort of patients to identify patterns (footprints) in a patient's gene expression profile that correspond to ex vivo drug resistance, or sensitivity. These patterns are specific to a given drug and cancer. Similarities between the patterns for any two drugs is estimated. Pairs of drugs that either have very similar patterns or complimentary patterns are identified to be ideal choices for a novel therapeutic strategy.

An example computer-implemented method for identifying gene signatures for therapy resistance is described herein. The method includes receiving patient data for a plurality of patients having a disease, where the patient data includes respective RNA sequencing data and respective ex vivo drug response data for the plurality of patients. The method also includes identifying one or more gene signatures for therapy resistance.

Additionally, the step of identifying one or more gene signatures for therapy resistance includes performing a cluster analysis.

Alternatively or additionally, the plurality of patients represent a heterogenous cohort of patients having early-, middle-, and late-stages of the disease.

Alternatively or additionally, the method further includes training a machine learning algorithm with a dataset created from the patient data and the identified one or more gene signatures for therapy resistance, where the machine learning model is configured to predict drug response. Additionally, the method further includes inputting, into the trained machine learning model, RNA sequencing data for a specific patient; and predicting, using the trained machine learning model, the specific patient's response to a drug.

Alternatively or additionally, the method further includes creating a gene regulatory network model with a dataset created from the patient data and the identified one or more gene signatures for therapy resistance, where the gene regulatory network model is configured to provide therapeutic strategies. Additionally, the method further includes inputting, into the gene regulatory network model, RNA sequencing data for a specific patient; and providing, using the gene regulatory network model, a therapeutic strategy for the specific patient. Optionally, the therapeutic strategy is a combination or sequential therapy.

A machine-learning based method for predicting drug response is also described herein. The method includes providing a trained machine learning model, where the trained machine learning model is configured to predict drug response. The method also includes inputting, into the trained machine learning model, RNA sequencing data for a specific patient. The method further includes predicting, using the trained machine learning model, the specific patient's response to a drug. Optionally, the method further includes administering the drug to the specific patient.

A method for modeling gene regulatory networks for providing therapeutic strategies is also described herein. The method includes providing a gene regulatory network model, where the gene regulatory network model is configured to provide therapeutic strategies. The method also includes inputting, into the gene regulatory network model, RNA sequencing data for a specific patient. The method further includes predicting, using the gene regulatory network model, a therapeutic strategy for the specific patient. Optionally, the method further includes administering the therapeutic strategy to the specific patient.

A method for identifying targeted therapies is also described herein. The method includes receiving patient data for a plurality of patients having a disease, where the patient data includes respective RNA sequencing data and respective ex vivo drug response data for the plurality of patients; and receiving RNA sequencing data for a specific patient. The method also includes deploying a trained machine learning model in response to the RNA sequencing data for the specific patient, where the trained machine learning model is configured to predict drug response; and deploying a gene regulatory network model in response to the RNA sequencing data for the specific patient, where the gene regulatory network model is configured to provide therapeutic strategies. Additionally, the method includes simulating, using a network controllability model, effects of a plurality of targeted therapies on the specific patient using the patient data, an output of the trained machine learning model, and an output of the gene regulatory network model. The method further includes predicting a drug response for the specific patient based on the simulation. Optionally, the method further includes administering the drug to the specific patient.

It should be understood that the above-described subject matter may also be implemented as a computer-controlled apparatus, a computer process, a computing system, or an article of manufacture, such as a computer-readable storage medium.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1: The figure depicts the use cases for the techniques described herein. The framework begins with a representative training cohort, whose biopsies are subjected to simultaneous RNA sequencing and ex vivo drug response estimation. The patient's gene expression profile is used to identify gene signatures implicated in therapy resistance, which is used to predict their sensitivity to standard-of-care (SOC) drugs by a machine learning model that is trained using matched gene expression and ex vivo drug sensitivity data from an independent training cohort. Further, a gene regulatory network (GRN) model is used to identify transcriptional regulatory mechanisms driving therapy resistance. These inferred mechanisms for various drugs are used to discover novel therapeutic strategies either as a combination, or sequential therapy. The gene signatures implicated in therapy resistance, machine learning model predictions of patient drug response from RNA sequencing data, and the gene regulatory network model to identify drivers of therapy resistance are all combined using a network controllability approach, where we simulate the effect of various targeted therapies on the patient's gene expression profile and predict a modified drug response for the patient using the machine learning model to identify targeted therapies that improve their response to SOC drugs.

FIG. 2: The bar plot shows the number of MM patients tested ex vivo with each single agent (in blue) and combination (in red). The cohort features a total of 500 patients tested with a total of 180 different single agents and 191 two-drug combinations.

FIG. 3: The figure presents an overview of the biomarker discovery tool, which begins with an MM biopsy from a patient that is subjected to RNA-sequencing and ex vivo drug sensitivity characterization through EMMA. The gene expression profile from 844 MM patients are used to obtain an MM transcriptomic map, which is a 2D visualization of co-expressing genes clustered together and identified as key myeloma gene programs (gene sets). Each of these gene sets are passed to GSEA along with matched ex vivo drug sensitivity data from EMMA and gene expression, to identify gene programs that are implicated in resistance (shown in red) and sensitivity (shown in green). All the genes implicated in resistance form a super cluster, representation of KEGG pathways and Cancer HALLMARKS is estimated using one-sided Fisher-exact tests to identify putative mechanisms of resistance to a SOC drug.

FIG. 4 validates the biomarker discovery tool by comparing the differentially enriched clusters using ex vivo drug sensitivity to differentially expressed genes in non-responders vs responders in two independent clinical trials in a Selinexor-based regimen. FIG. 6a presents MM gene programs enriched for resistance (in red) and sensitivity (in blue) as identified by the biomarker discovery tool. FIGS. 4b and 4c show genes differentially expressed in non-responders in red and those differentially expressed in responders in blue observed in two independent clinical trials on an arm involving a Selinexor-based regimen.

FIG. 5: The figure shows a flowchart used to train a regression tree model using genes implicated in resistance and sensitivity to Selinexor across a large cohort as inputs and ex vivo drug sensitivity metric AUC as an output. The model predicted ex vivo drug sensitivity (AUC) is correlated with actual aucs of the patients to show strong correlation between the two. Furthermore, the model predicted aucs are used as an accurate classifier to distinguish between responders and non-responders with an AUC of ROC curve of 0.7544.

FIG. 6A shows the gene clusters on the MM transcriptomic map that are enriched for resistance (red) and sensitivity (green) to bortezomib tested ex vivo in more than 400 patient samples.

FIGS. 6B and 6C show the gene clusters on the MM transcriptomic map that are enriched for resistance (red) and sensitivity (green) to daratumumab tested ex vivo in around 250 patient samples.

FIG. 6D shows the gene clusters on the MM transcriptomic map that are enriched for resistance (red) and sensitivity (green) to Selinexor. The figure also lists all the KEGG pathways and cancer hallmarks implicated in resistance, or sensitivity to daratumumab. It is important to note the complementary nature in the transcriptomic maps for resistance to both bortezomib and daratumumab, and daratumumab and selinexor, which is further exemplified by the complementary pathways implicated for resistance and sensitivity by GSEA.

FIGS. 7A-7G: The figure presents an overview of the gene regulatory network modeling approach beginning with an MM transcriptomic landscape constructed by carrying out dimensionality reduction (in FIG. 7A) using 844 patient gene expression data, where every point represents a gene. Genes that are closer to each other are said to co-express and form a gene cluster that putatively represent an MM gene program. These co-expressing genes are clustered together using fuzzy c-means clustering (in FIG. 7B). Each of the colored gene clusters form the foundation of a three-layer GRN as depicted in FIG. 7C. Upstream tfs that regulate the genes in the cluster are identified by computing a representation factor for the gene cluster among all the genes bound by the transcription factor using publicly available data from genome-wide chip-X experiments. Further, kinases that can phosphorylate upstream tfs are identified using publicly available proteomic databases like phosphositeplus and phosphopoint. The functional relationship between target genes, tfs, and kinases is given by a biophysical model accounting for post-translational modifications, translation, and transcription in FIGS. 7D, 7E, and 7F. These system of equations under steady-state are one given by FIG. 7G.

FIG. 8A correlated the model predicted gene expression to the actual gene expression within the training cohort. This plot shows how well the model allows for fitting expression data.

FIG. 8B correlates the model predicted gene expression to actual gene expression within the validation cohort. This plot shows how well the model predicts gene expression.

FIG. 8C highlights genes (shown in red) that have a validation correlation greater than 0.5.

FIG. 8D highlights genes (shown in yellow) that have a significant variability in chromatin accessibility. These two plots show that there is a near-perfect complimentary overlap, implying that the GRN model predicts gene expression accurately if there is no significant variation in chromatin accessibility.

FIG. 9 shows a clustergram of resensitization achieved by a candidate resensitizer (columns) working to overcome, or dealy proteasome inhibitor resistance for each patient (rows). Blue indicates a great benefit in resensitization by the corresponding resensitizer (column) for that patient (row), while red implies a poor resensitization effect. The targets for each cluster of kinase inhibitors (dendrogram of columns) are also labeled.

FIG. 10A presents a clustergram of normalized enrichment scores (NES) computed using Cancer Hallmarks as supervised gene sets, where NES represent the enrichment of a Cancer Hallmark by overexpression of genes implicated in resistance (red), or underexpression of genes implicated in sensitivity (blue) using GSEA. Similarly, Supplementary FIG. 10B is a clustergram that uses KEGG Pathways as the supervised gene sets to carry out GSEA.

FIG. 11A presents the MM transcriptomic landscape identified by carrying out dimensionality reduction using tsne on normalized gene expression data from RNA-seq.

FIG. 11B presents clusters of co-expressing genes identified by fuzzy c-means clustering, which serve as MM-specific gene programs.

FIG. 11C highlights gene programs that are enriched for resistance in red and sensitivity in blue using GSEA.

FIGS. 11D-11E present bubble plots showing combined enrichr score for sensitivity in blue and resistance in red, with the size of the bubble signifying the p-value of the enrichment as identified by a one-sided Fisher exact test.

FIGS. 12A and 12B present enriched gene programs for clinical resistance in red and sensitivity in blue for BOSTON and MCC17814 using GSEA and paired gene expression data and clinical response.

FIGS. 12C and 12D present bubble plots showing enriched epigenetic alterations and transcription factors using clinical response data from BOSTON and MCC17814 clinical trials with the red labels indicating the transcription factors/epigenetic alterations identified using ex vivo drug sensitivity data.

FIG. 12E compares the predicted ex vivo AUC of BOSTON trial patients from their gene expression data alone between responders and non-responders.

FIG. 12F uses the predicted ex vivo response (AUC) as a classifier to identify the patients' response as a responder, or non-responder in the BOSTON trial using a ROC curve.

FIG. 12G presents the predicted ex vivo drug response data shown in 12E but classifies them based on predicted AUC itself into AUC high and AUC low groups.

FIG. 12H compares the probability of survival by PFS between the AUC high and AUC low groups identified in FIG. 12G.

FIG. 13A shows a clustergram of correlations of NES scores for each cluster for every pair of drugs tested ex vivo.

FIG. 13B presents a clustergram of enriched cancer hallmarks for ex vivo drug sensitivity/resistance to Selinexor and Daratumumab. FIGS. 13C and 13D present the anti-correlative transcriptomic profiles of Selinexor and Daratumumab.

FIGS. 13E and 13F compare response rates between patients who received an anti-CD38 monoclonal antibody as an immediate prior line to a Selinexor-based treatment (yellow) versus those who didn't in STOMP and XPORT-MM-028.

FIGS. 13G and 13H compare the probability of survival by PFS for the two groups and show a significant improvement of survival in patients treated with a Selinexor-based regimen with a Daratumumab based regimen as an immediate prior line in STOMP and XPORT-MM-028.

FIG. 14: The use of a computational framework to identify differentially expressed gene clusters based on resistance to Selinexor highlighted in red and sensitivity to Selinexor highlighted in green.

FIG. 15: A volcano plot showing the extent of synergy in terms of the log 2 fold-change over an additive response is shown along the x-axis and the likelihood of synergy within the patient cohort as −log 10 p-value is shown along the y-axis for the combinations of Selinexor and dexamethasone KPT+DEX) Selinexor and pomalidomide (KPT+POM), Selinexor and elotuzumab (ELOTUZUMAP+KPT), and Selinexor and daratumumab (DARATUMUMAB+KPT).

FIGS. 16A-16D are transcriptomic maps highlighting clusters based on intensity of synergism (red) and antagonism (blue) for Selinexor and dexamethasone (FIG. 16A) Selinexor and pomalidomide (FIG. 16B), Selinexor and elotuzumab (FIG. 16C), and Selinexor and daratumumab (FIG. 16D).

FIGS. 17A-17D show box and whisker plots to show the extent of ex vivo synergy for Selinexor and dexamethasone (FIG. 17A) Selinexor and pomalidomide (FIG. 17B), Selinexor and elotuzumab (FIG. 17C), and Selinexor and daratumumab (FIG. 17D), where the first and fourth columns show LD₅₀s for the two single agents, the second column shows the LD₅₀ for the additive response, and the third column shows the LD₅₀s for the combination. Each line represents a patient, red indicates synergy and blue indicates antagonism.

FIG. 18: A box and whisker plot showing the extent of ex vivo synergy for the combination Selinexor, Dexamethasone, and Daratumumab.

FIG. 19 is an example computing device.

FIG. 20 is a table with examples of transcription factors implicated in resistance and sensitivity to various drugs.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. Include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) Having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. A composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Method of Treating Cancer and Assessing Patient Responsiveness to a Therapeutic Regimen

We developed a novel ex vivo assay (EMMA—Ex vivo Mathematical Myeloma Advisor) that can extend overall patient survival by using the most effective drugs and withdrawing agents with no clinical benefit. In this assay, patient-derived primary MM cells are co-cultured in an ex vivo reconstruction of the tumor microenvironment and treated with standard-of-care (SOC) drugs. A non-destructive, bright-field-based image analysis algorithm estimates percent viability across time and concentration, and these data parameterize tumor/drug mathematical models of drug sensitivity. These models are coupled with pharmacokinetic data from Phase I clinical trials to predict a patient's response to single agents and combinations. The proposed ex vivo framework is used to predict a patient's early objective response (EOR) to each of the drugs tested and quantify synergistic effects in combinations that can translate into the clinic. Even though EOR predictions play a quintessential role in choosing the best therapeutic option, evolution of therapy resistance eventually leads to a multi-drug resistant state. Identifying patient-specific therapeutic strategies to evade or overcome therapy resistance from a patient's gene expression profile alone can significantly improve overall survival and provide novel effective therapeutic options.

Disclosed herein are methods of measuring tumor chemosensitivity in a subject with multiple myeloma comprising obtaining multiple myeloma cells from a subject; culturing said multiple myeloma cells; contacting the multiple myeloma cells with one or more individual anti-cancer agents and/or combinations of two or more anti-cancer agents; taking an image (such as, for example a bright field image) of said multiple myeloma cells at least two times; and applying an image analysis algorithm to said images to determine viability (including, but not limited to assessing non-translational cellular membrane motion (i.e., undulations of the cell membrane that are not caused by a cell moving from point a to point b, but a change in cell membrane motion and/or integrity) of the cell after stage drift and field vibrations are excluded) across time and/or concentration thereby forming a model of drug sensitivity.

In one aspect, disclosed herein are methods of measuring tumor chemosensitivity, wherein the multiple myeloma cells are cultured in the presence of stroma, collagen, and/or plasma.

Also disclosed herein are methods of measuring tumor chemosensitivity, wherein at least one image is obtained prior to the contact with the anti-cancer agent

It is understood and herein contemplated that the multiple myeloma cells can be imaged for at least 2, 3, 4, 5, 6, 7, 8, 9, 10 11, 12, 13, 14, 15, 16, 17, 18, 19, 20, 21, 22, 23, 24, 25, 26, 27, 28, 29, 30, or 31 days and/or wherein images taken of the multiple myeloma cells every 5, 10, 15, 20, 25, 30, 35, 40, 45, 50, 55, 60, 65, 70, 75, 80, 85, 90, 95, 100, 105, 110, 115, 120 min, 3, 4, 5, 6, 7, 8, 9, 10, 11, 12, 13, 14, 15, 16, 17, 18, 19, 20, 21, 22, 23, 24, 36, 48, 60, 72 hours.

To assess anti-cancer agent sensitivity, the multiple myeloma cells can be separately contacted with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more individual anti-cancer agents and/or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, or more combinations of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 individual anti-cancer agents.

It is understood and herein contemplated that one result of the assays disclosed herein is the ability to combine model data with clinical trial information to establish a patient's early objective response (EOR) to each of the drugs tested and quantify synergistic effects in combinations and/or compare with other patients to establish drug sensitivities and synergistic effects across a cohort. Thus, for example, disclosed herein are methods of measuring tumor chemosensitivity further comprising coupling drug sensitivity model to clinical trials to establish a patient's response to single agents and combinations thereby establishing a patient's early objective response (EOR) to each of the drugs tested and quantify synergistic effects in combinations.

It is understood that the therapeutic benefit of applying the measurements and analysis disclosed herein is to establish an effective treatment regimen for a patient. Thus, also disclosed herein are methods of measuring tumor chemosensitivity, further comprising applying or adjusting a patient's treatment regimen based on the sensitivities.

In one aspect, disclosed herein are methods of identifying gene signatures associated with anti-cancer therapy resistance for multiple myeloma comprising obtaining multiple myeloma cells from a subject; culturing said multiple myeloma cells; contacting the multiple myeloma cells with one or more individual anti-cancer agents and/or combinations of two or more anti-cancer agents; taking an image of said multiple myeloma cells at least two times; applying an image analysis algorithm to said images to determine viability across time and/or concentration thereby forming a model of drug sensitivity; in parallel to assaying cellular sensitivity to one or more anti-cancer agents, sequencing multiple myeloma cells obtained from the patient; analyzing the gene expression profiled obtained from the sequence information in combination with the drug sensitivity data to determine gene signatures associated with therapy resistance.

Also disclosed herein are methods of identifying gene signatures associated with anti-cancer therapy resistance, further comprising applying gene signature data to a gene regulatory network (GRN) model to identify transcriptional regulatory mechanisms driving therapy resistance.

In one aspect, also disclosed herein are methods of identifying gene signatures associated with anti-cancer therapy resistance, further comprising repeating the analysis from two or more patients to identify gene signatures and/or transcriptional regulatory mechanisms driving therapy resistance common to a cohort.

Also disclosed herein are methods of identifying therapeutic regimens for a subject comprising identifying gene signatures using the method disclosed herein and identifying transcriptional regulatory mechanisms driving therapy resistance using the method disclosed herein; applying gene signature and GRN model information to identify novel therapeutic strategies either as a combination, or sequential therapy.

In one aspect, disclosed herein are methods of identifying novel therapeutic regimens for the treatment of multiple myeloma comprising identifying gene signatures using the method disclosed herein from two or more patients and identifying transcriptional regulatory mechanisms driving therapy resistance using the method disclosed herein from two or more patients; applying gene signature and GRN model information to identify novel therapeutic strategies either as a combination, or sequential therapy.

The disclosed methods can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, multiple myeloma, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

### Example Methods

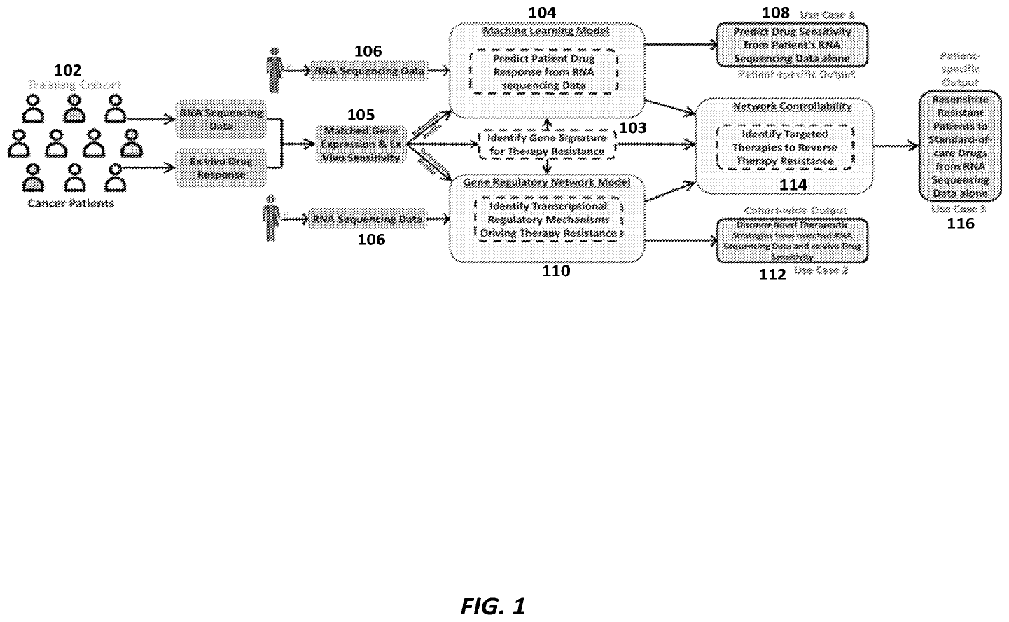

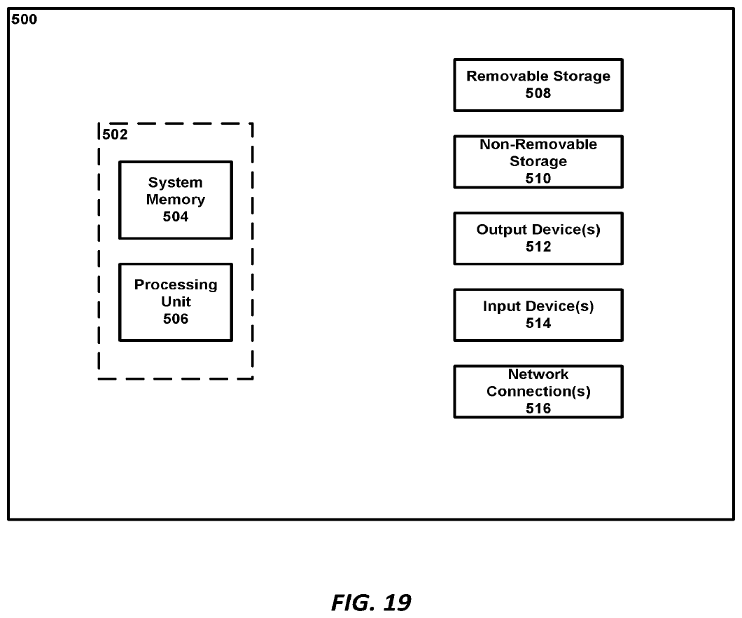

Referring now to FIG. 1, a method for identifying gene signatures for therapy resistance is described. As described herein, this disclosure contemplates that logical operations can be performed using a computing device (e.g., a computing device as shown in FIG. 19). The method includes receiving patient data 102 for a plurality of patients having a disease. Optionally, the disease is multiple myeloma. It should be understood that multiple myeloma is provided only as an example. This disclosure contemplates that the disease may be another cancer or another type of disease. The patient data 102 includes respective RNA sequencing data and respective ex vivo drug response data for the plurality of patients. As described herein, this data can be obtained from biopsies of patient samples that are simultaneously subjected to RNA sequencing and ex vivo drug response estimation. Optionally, the plurality of patients represent a heterogenous cohort of patients having early-, middle-, and late-stages of the disease. The method also includes identifying one or more gene signatures for therapy resistance.

As described in the Examples below, to unravel the transcriptomic topology of a complex disease like MM, RNA-seq data from 844 patients was employed to identify modules of co-expressing genes using a robust dimensionality reduction technique and an efficient clustering method. Z-normalized expression of 16,738 genes across 844 MM patients is used to identify groups of co-expressing genes that are likely to play disease-specific functional roles. Although it is typical to consider genes as variables (dimensions) and patients as observations (typically used with single-cell sequencing data to identify clones, or cell types), patients are instead perceived as variables uniquely contributing to a high-dimensional MM heterogeneity space and genes as observations that govern MM transcriptomic topology. This leaves us with 16,738 genes spread across a massively high-dimensional (844) MM patient space. This high-dimensional data is projected onto a two-dimensional space⁴⁵ using t-Student Neighbor Embedding (tsne)⁴⁶, a dimensionality reduction technique known in the art that specializes in extracting features (co-expression of genes) that lie on various low-dimensional embedded manifolds⁴⁶, thereby serving as an excellent visualization tool depicting a disease-specific two-dimensional transcriptomic map. Locations of genes on this 2D map are used to identify functional modules by employing an efficient clustering algorithm called fuzzy C-means⁴⁷, which results in 500 distinct gene clusters (gene sets) of varying sizes.

The ex vivo drug sensitivity data for each drug from EMMA is used to identify clusters that are enriched for resistance and sensitivity using gene set enrichment analysis (GSEA)⁴⁸. GSEA estimates an enrichment score for each cluster (gene set) using a running-sum statistic along the ranked-list of all genes (16,738) based on the correlation between their expression and the continuous phenotypic variable (AUC). Whenever GSEA encounters a gene that belongs to the cluster, it increases the running-sum statistic and decreases it, if it doesn't encounter a gene from that cluster. The maximum value of this running-sum statistic is the enrichment score for that cluster associated with positive correlation to the continuous phenotypic variable. GSEA estimates the statistical significance of such an enrichment by randomly scrambling the phenotypic variable several times and for each case generates a ranked gene list and the corresponding enrichment score for the cluster of interest. All these enrichment scores form a null distribution, which is compared to the enrichment score for the cluster using the actual input data to estimate the nominal p-value of enrichment. This approach is repeated for all 500 clusters and their nominal p-values are corrected for multiple hypothesis testing. The clusters that are enriched for resistance and sensitivity are identified by a family-wise error rate that is less than 5%. These enriched clusters, collectively, form the transcriptomic footprint of the drug in MM. This disclosure contemplates performing this step using the techniques described herein, e.g., see Examples. Optionally, this step includes performing a cluster analysis.

The method further includes training a machine learning model 104 with a dataset created from the patient data 102 and the identified one or more gene signatures for therapy resistance 103. Machine learning models include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model learns a function that maps an input (also known as feature or features) to an output (also known as target or target) during training with an unlabeled data set. In a semi-supervised model, the model learns a function that maps an input (also known as feature or features) to an output (also known as target or target) during training with both labeled and unlabeled data.

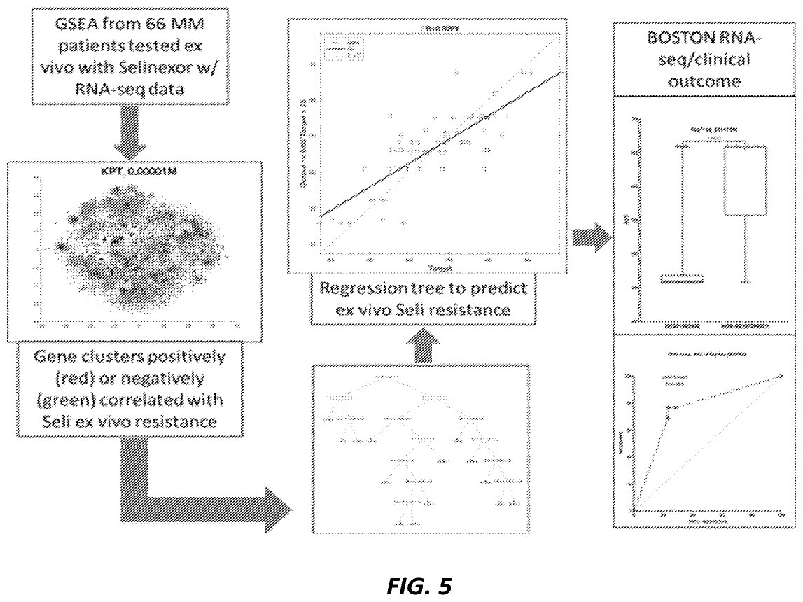

In the examples described herein, the machine learning model 104 is either a regression tree-based model (i.e., a supervised learning model), or a convoluted neural network model (CNN). An example training process for the regression tree-based model is described in detail below with regard to FIG. 5, for example. A regression tree model was used to estimate a molecular biomarker score from the median expression of the genes in each enriched cluster of the transcriptomic footprint as inputs to the model. The model was trained using the extensive paired ex vivo AUC response as output and RNA-seq data as input, where the predicted ex vivo AUC response from gene expression data would serve as a surrogate for clinical response in MM. The regression tree modeling was implemented in Matlab computational environment using the regression tree class available in the Statistics and Machine Learning Toolbox. It should be understood that a regression tree-based model is provided only as an example. This disclosure contemplates using other machine learning models with the techniques described herein including, but not limited to artificial neural networks.

An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan h, or rectified linear unit (relu) function), and provide an output in accordance with the activation function. Additionally, each node is associated with a respective weight. Anns are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for anns include, but are not limited to, backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

As noted above, the machine learning model 104 is trained to learn a function that maps an input (also known as feature or features) to an output (also known as target or target). In the examples described herein, the model features and target include matched gene expression and ex vivo drug sensitivity data 105. Once trained, the machine learning model 104 is configured to predict a patient's drug response (target) based on RNA sequencing data (features).

Additionally, this disclosure contemplates using the trained machine learning model 104 in inference mode. In particular, as shown in FIG. 1, the method further includes inputting, into the trained machine learning model 104, RNA sequencing data for a specific patient 106; and predicting, using the trained machine learning model 104, the specific patient's response 108 to a drug.

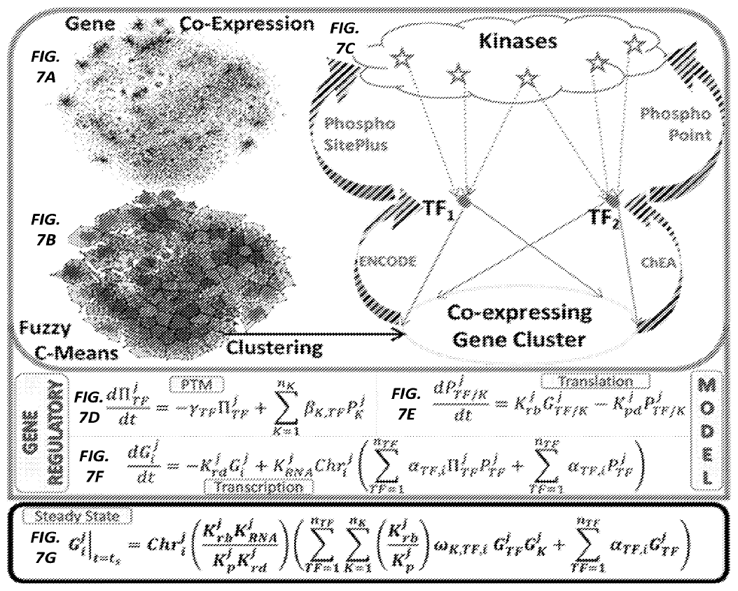

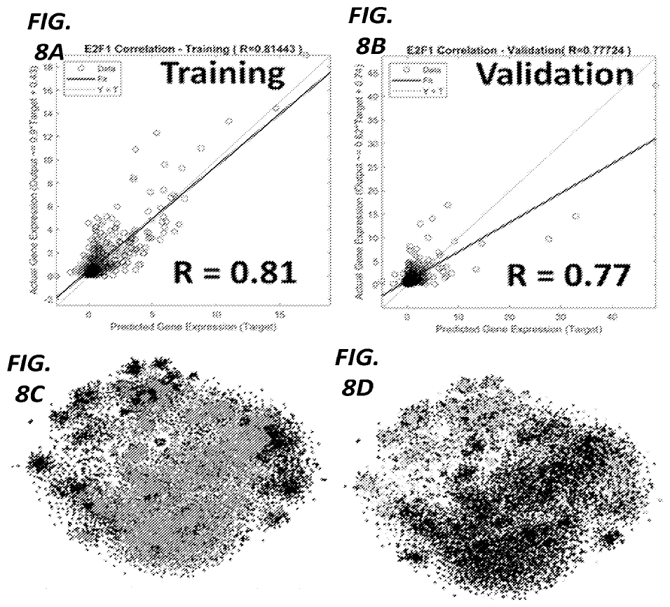

The method further includes creating a gene regulatory network model 106 with a dataset created from the patient data 102 and the identified one or more gene signatures for therapy resistance 103. An example gene regulatory modelling approach is described in detail below with regard to FIGS. 7A-7G, for example. The gene regulatory network model 106 is configured to provide therapeutic strategies that reverse resistance to an SOC drug. This is particularly useful for patients with late-stage disease, who tend to acquire multi-drug resistance. With dwindling therapeutic options at this juncture, reversing therapy resistance to an SOC drug remains to be the only viable option. To this end, a gene regulatory network was constructed for each gene cluster featuring co-expressing genes in MM. These genes likely co-express in MM patients as they are regulated by a common transcription factor that plays an important role in MM pathogenesis. Transcription factors regulating the genes in a cluster are identified from publicly available databases with genome-wide ChIP-X data linking transcription factors and the genes these transcription factors bind to. Further, these transcription factors are activated by kinases that phosphorylate them, which are identified from publicly available databases featuring phosphor-proteomic data like PhosphoSitePlus and PhosphoPoint. A considerable number of these kinases are druggable targets, thereby allowing a therapeutic intervention to selectively target the transcription factor that is regulating a cluster of genes that are implicated in a resistance mechanism. Such a cascading network of genes implicated in resistance mechanisms, transcription factors that regulate these genes, and kinases that phosphorylate them are formed into a gene regulatory network. The edges connecting any two nodes of such a network represent the selective influence of the upstream transcription factor, or kinase on that gene. This is quantified using a biophysical model described in detail in Example 5. The biophysical model is parametrized by a patient's RNA sequencing data, thereby resulting in a GRN model for each patient and gene cluster. The biophysical model is simulated to quantify the effect of kinase inhibition by silencing the effect of the kinase inhibitor (by setting the variable equal to zero), this is done for each cluster identified in the transcriptomic footprint of an SOC drug. The silencing of the kinase leads to a modified gene expression profile in these clusters, which is fed to the machine learning model to predict modified ex vivo AUC of an SOC drug due to the kinase inhibition. This typically leads to a lower AUC (sensitivity to SOC drug), as the genes and mechanisms overexpressed in resistance (or under-expressed in sensitivity) are suppressed (or activated) by kinase inhibition. Therefore, adding a kinase inhibitor to the SOC drug regimen, could reverse resistance to therapy and prolong survival.

Additionally, this disclosure contemplates using the gene regulatory network model 106 in inference mode. In particular, as shown in FIG. 1, the method further includes inputting, into the gene regulatory network model 106, RNA sequencing data for a specific patient 106; and providing, using the gene regulatory network model 106, a therapeutic strategy 112 for the specific patient. Optionally, the therapeutic strategy 112 is a combination or sequential therapy.

Alternatively or additionally, a machine-learning based method for predicting drug response is also described herein. The method includes providing a trained machine learning model 104, where the trained machine learning model 104 is configured to predict drug response. The method also includes inputting, into the trained machine learning model 104, RNA sequencing data for a specific patient 106. The method further includes predicting, using the trained machine learning model 104, the specific patient's response 108 to a drug. Optionally, the method further includes administering the drug to the specific patient, for example, when the machine learning model 104 predicts a positive drug response for the patient.

Alternatively or additionally, a method for modeling gene regulatory networks for providing therapeutic strategies is also described herein. The method includes providing a gene regulatory network model 106, where the gene regulatory network model 106 is configured to provide therapeutic strategies. The method also includes inputting, into the gene regulatory network model 106, RNA sequencing data for a specific patient 106. The method further includes predicting, using the gene regulatory network model 106, a therapeutic strategy 112 for the specific patient. Optionally, the method further includes administering the therapeutic strategy 112 to the specific patient.

Alternatively or additionally, a method for identifying targeted therapies is also described herein. The method includes receiving patient data 102 for a plurality of patients having a disease, where the patient data 102 includes respective RNA sequencing data and respective ex vivo drug response data for the plurality of patients; and receiving RNA sequencing data for a specific patient 106. The method also includes deploying a trained machine learning model 104 in response to the RNA sequencing data for the specific patient, where the trained machine learning model 104 is configured to predict drug response; and deploying a gene regulatory network model 106 in response to the RNA sequencing data for the specific patient, where the gene regulatory network model 106 is configured to provide therapeutic strategies. Additionally, the method includes simulating, using a network controllability approach 114, effects of a plurality of targeted therapies on the specific patient using the patient data 102, an output of the trained machine learning model 104, and an output of the gene regulatory network model 106. The method further includes predicting a drug response for the specific patient based on the simulation. This may include identifying a targeted therapy 116 to reverse the specific patient's resistance to therapy. Optionally, the method further includes administering the drug and/or targeted therapy to the specific patient.

### Example Computing Device

It should be appreciated that the logical operations described herein with respect to the various figures may be implemented (1) as a sequence of computer implemented acts or program modules (i.e., software) running on a computing device (e.g., the computing device described in FIG. 19), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 19, an example computing device 500 upon which the methods described herein may be implemented is illustrated. It should be understood that the example computing device 500 is only one example of a suitable computing environment upon which the methods described herein may be implemented. Optionally, the computing device 500 can be a well-known computing system including, but not limited to, personal computers, servers, handheld or laptop devices, multiprocessor systems, microprocessor-based systems, network personal computers (pcs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 500 typically includes at least one processing unit 506 and system memory 504. Depending on the exact configuration and type of computing device, system memory 504 may be volatile (such as random access memory (RAM)), non-volatile (such as read-only memory (ROM), flash memory, etc.), or some combination of the two. This most basic configuration is illustrated in FIG. 19 by dashed line 502. The processing unit 506 may be a standard programmable processor that performs arithmetic and logic operations necessary for operation of the computing device 500. The computing device 500 may also include a bus or other communication mechanism for communicating information among various components of the computing device 500.

Computing device 500 may have additional features/functionality. For example, computing device 500 may include additional storage such as removable storage 508 and non-removable storage 510 including, but not limited to, magnetic or optical disks or tapes. Computing device 500 may also contain network connection(s) 516 that allow the device to communicate with other devices. Computing device 500 may also have input device(s) 514 such as a keyboard, mouse, touch screen, etc. Output device(s) 512 such as a display, speakers, printer, etc. May also be included. The additional devices may be connected to the bus in order to facilitate communication of data among the components of the computing device 500. All these devices are well known in the art and need not be discussed at length here.

The processing unit 506 may be configured to execute program code encoded in tangible, computer-readable media. Tangible, computer-readable media refers to any media that is capable of providing data that causes the computing device 500 (i.e., a machine) to operate in a particular fashion. Various computer-readable media may be utilized to provide instructions to the processing unit 506 for execution. Example tangible, computer-readable media may include, but is not limited to, volatile media, non-volatile media, removable media and non-removable media implemented in any method or technology for storage of information such as computer readable instructions, data structures, program modules or other data. System memory 504, removable storage 508, and non-removable storage 510 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 506 may execute program code stored in the system memory 504. For example, the bus may carry data to the system memory 504, from which the processing unit 506 receives and executes instructions. The data received by the system memory 504 may optionally be stored on the removable storage 508 or the non-removable storage 510 before or after execution by the processing unit 506.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-roms, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### C. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Ex Vivo Drug Sensitivity Estimation

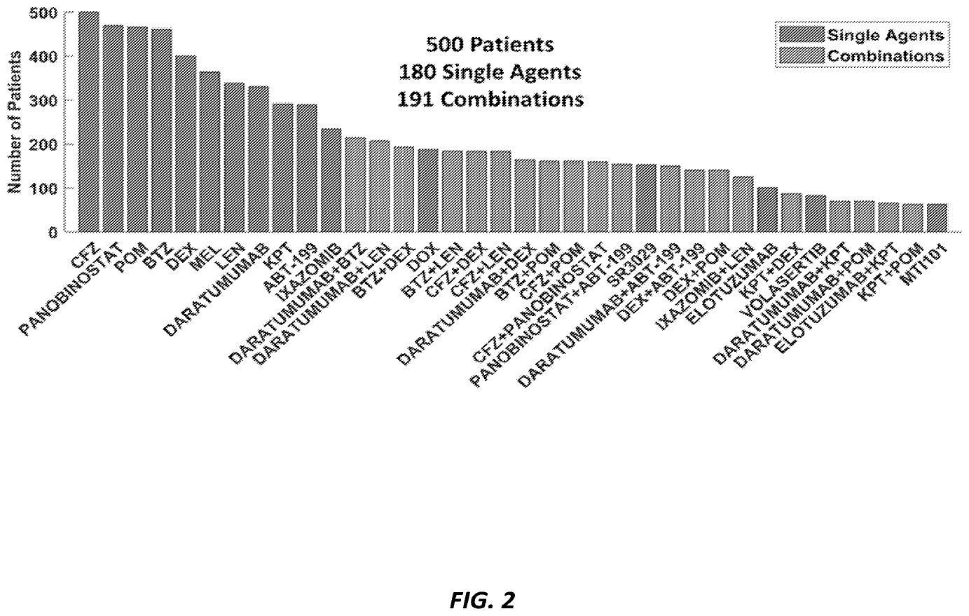

There is a dire need for clinical decision support tools that aid physicians in their effort to increase patient survival and improve quality of life, thus avoiding emergence of multi drug resistance. Such tools would need to characterize resistance to therapy for each class of drugs and individual patients before identifying ways to evade it. Since MM patients receive combination therapies, it is not possible to quantify resistance to single agents using clinical response data. Hence, it is imperative that a patient's drug sensitivity be estimated in a controlled environment, where resistance to each drug is quantified independently. To this end, our ex vivo assay (EMMA—Ex vivo Mathematical Malignancy Advisor) serves as an ideal framework to characterize patient-specific drug sensitivity/combination effect from patient-derived MM cells. EMMA estimates a patient's tumor chemosensitivity through live imaging of a co-culture of bone-marrow derived MM cells with human stroma, collagen and patient-derived plasma, in 384-well or 1,536-well plates (31 or 126 drug/drug combinations simultaneously) every 30 minutes, for six days. Each patient sample is tested with 31 single agents or combinations, across five serially diluted concentrations (1:3 ratio), in duplicates. An image-processing algorithm is employed to estimate the percent viable cells for each of these experimental conditions (drug, concentration, time point) using exclusively cell membrane motion calculated from sequences of brightfield images. The percent viability measures are fit to mathematical models of varying phenotypic heterogeneity and the patient-drug specific sensitivity are characterized using LD50 (dose for median effect) and AUC (area under the curve). Combination effect is quantified using ex vivo response for the two single agents and their combination to estimate the percent reduction in viability over a computed (from the two single-agent responses) additive response. We utilized this framework to quantify ex vivo drug sensitivity/combination effect for 500 MM patients with a total of 180 drugs and 191 combinations, where many SOC drugs were tested with over 300 samples and several SOC combinations were tested with over 150 samples, as depicted in FIG. 2. To the best of our knowledge, this is the most comprehensive and extensive MM patient drug sensitivity database developed so far.

### 2. Example 2: Biomarker Discovery Tool

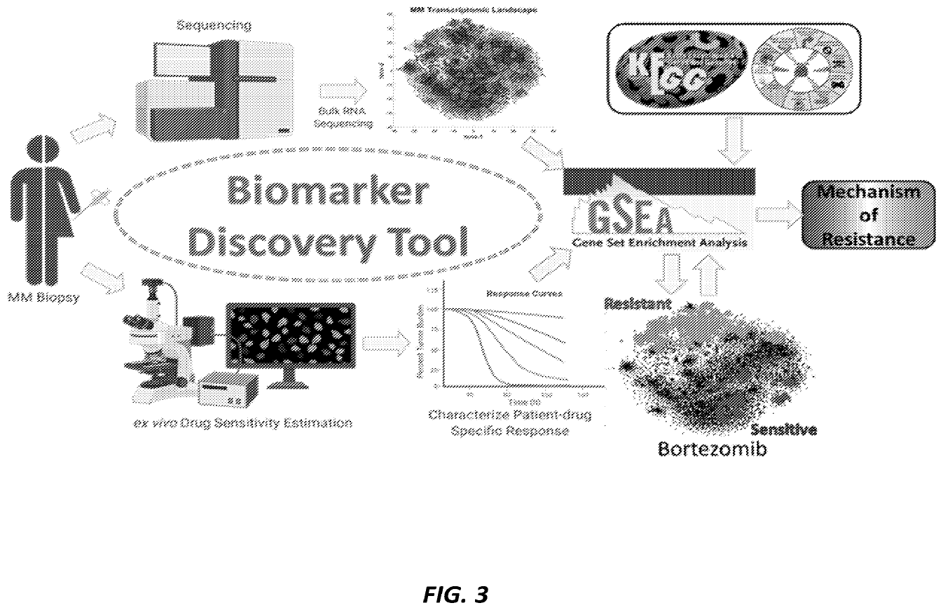

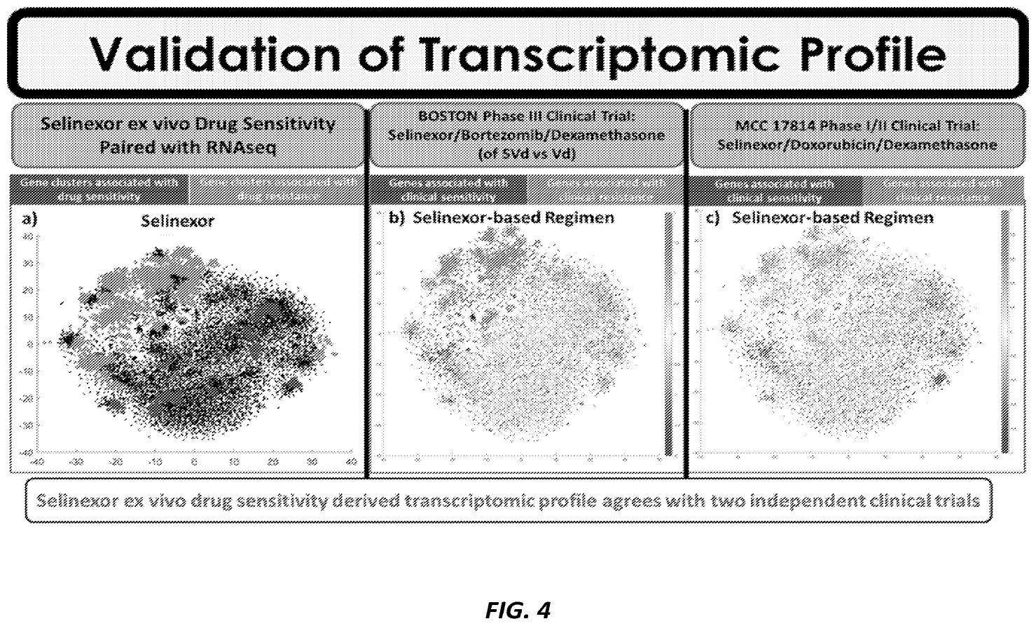

Standard-of-care treatments received by an MM patient involve drugs from various classes such as proteasome inhibitors, immunomodulatory agents, corticosteroids, and more recently, targeted therapies (e.g. BCL2 inhibitors) and monoclonal antibodies (e.g. Daratumumab). Given the broad spectrum of the mechanism of action of some of these drugs, and inherent inter-patient tumor molecular heterogeneity, it is to be expected that patients' mechanisms of resistance towards multidrug therapies can be extremely diverse, and hard to capture using single-omic biomarkers. In order to unravel the transcriptomic topology of a complex disease like MM, we employed RNA-seq data from 844 patients to identify modules of co-expressing genes using a robust dimensionality reduction technique and an efficient clustering method. Z-normalized expression of 16,738 genes across 844 MM patients is used to identify groups of co-expressing genes that are likely to play disease-specific functional roles. Although it is typical to consider genes as variables (dimensions) and patients as observations (typically used with single-cell sequencing data to identify clones, or cell types), we instead perceive patients as variables uniquely contributing to a high-dimensional MM heterogeneity space and genes as observations that govern MM transcriptomic topology. This leaves us with 16,738 genes spread across a massively high-dimensional (844), MM patient space. We project this high-dimensional data onto a two-dimensional space using t-Student Neighbor Embedding (tsne), a well-known dimensionality reduction technique that specializes in extracting features (co-expression of genes) that lie on various low-dimensional embedded manifolds, thereby serving as an excellent visualization tool depicting a disease-specific two-dimensional transcriptomic map. Locations of genes on this 2D map are used to identify functional modules by employing an efficient clustering algorithm called fuzzy C-means, which results in 500 distinct gene clusters (gene sets) of varying sizes. We use the ex vivo drug sensitivity data for each drug from EMMA, to identify clusters that are enriched for resistance and sensitivity using gene set enrichment analysis (GSEA). GSEA estimates an enrichment score for each cluster (gene set) using a running-sum statistic along the ranked-list of all genes (16,738) based on the correlation between their expression and the continuous phenotypic variable (AUC). Whenever GSEA encounters a gene that belongs to the cluster, it increases the running-sum statistic; and decreases it, if it doesn't encounter a gene from that cluster. The maximum value of this running-sum statistic is the enrichment score for that cluster associated with positive correlation to the continuous phenotypic variable. GSEA estimates the statistical significance of such an enrichment by randomly scrambling the phenotypic variable several times and for each case generates a ranked gene list and the corresponding enrichment score for the cluster of interest. All these enrichment scores form a null distribution, which is compared to the enrichment score for the cluster using the actual input data to estimate the nominal p-value of enrichment. This approach is repeated for all the 500 clusters and their nominal p-values are corrected for multiple hypothesis testing. The clusters that are enriched for resistance and sensitivity are identified by a false discovery rate that is less than 5%. The genes in these clusters not only correlate with resistance/sensitivity, they also co-express among themselves, thereby implicating them in a pathway/mechanism, or a common upstream regulator. We ascertain if genes implicated in resistance/sensitivity to a drug in MM are associated with any of the known pathways/mechanisms listed in Kyoto Encyclopedia of Genes and Genomes (KEGG) and cancer hallmarks using one-sided Fisher exact tests to identify biomarkers for resistance to therapy in MM using GSEA. An overview of this Biomarker Discovery Tool is presented in FIG. 3. We validated this approach in two independent clinical trials involving a Selinexor-based regimen, where we compared the gene expression of responders with non-responders and highlighted genes implicated in resistance in red and sensitivity in blue. FIG. 4 shows the genes implicated in clinical resistance or sensitivity match the corresponding gene programs identified by the biomarker discovery tool.

### Functional Transcriptomic Landscapes in MM Identifies Gene Expression Footprints for Drug Sensitivity:

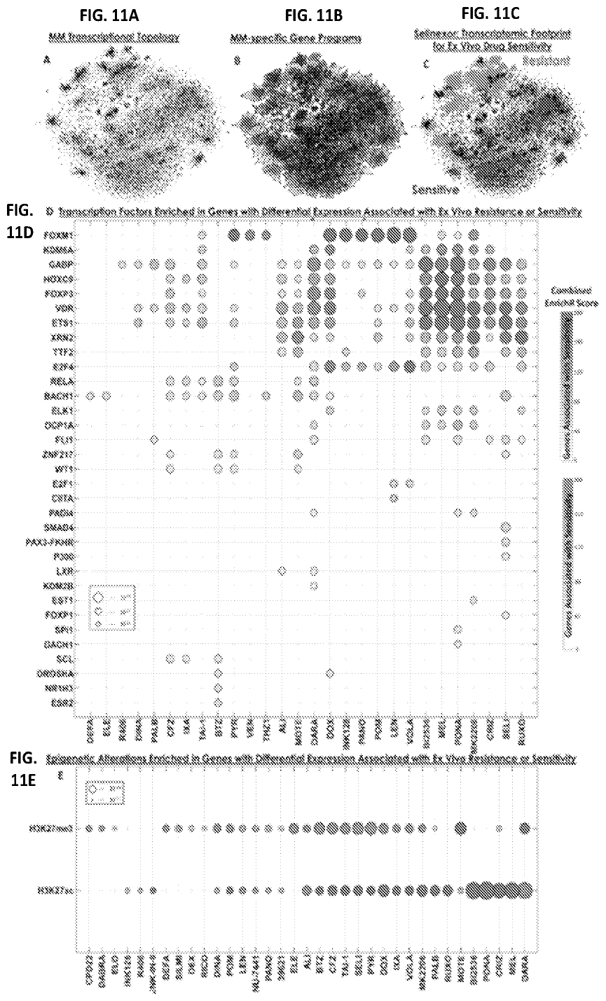

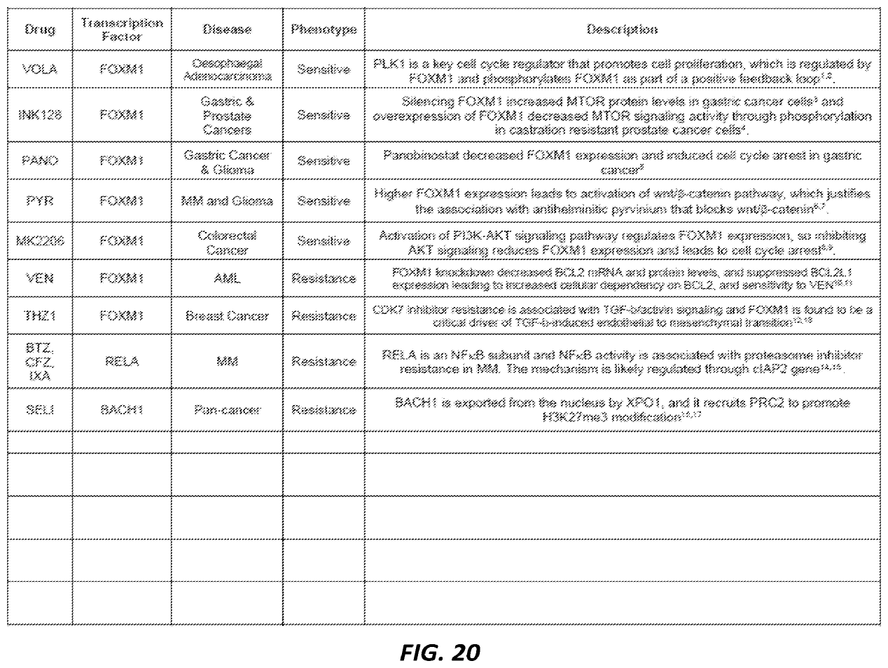

Gene set enrichment analysis (GSEA) was carried out using paired z-normalized RNA-seq data and ex vivo drug sensitivity measures (AUC) for each drug that was tested in more than 20 samples, to identify cancer hallmarks and KEGG pathways enriched for sensitivity (blue) and resistance (red), which resulted in FIGS. 10A and 10B respectively. From the two clustergrams, we noticed that the proteasome inhibitors (PI) BTZ, CFZ, and Ixazomib (IXA) do not enrich any common cancer hallmarks, or KEGG pathways; with CFZ being the only PI that overexpresses the proteasome KEGG pathway in CFZ resistant patients. Also, MK2206 (AKT Inhibitor) and INK128 (MTOR Inhibitor) are not enriched for the cancer hallmark PI3K-AKT MTOR signaling. We expect that this is due to the relatively poor co-expression of genes identified in these supervised gene sets in MM patient samples. To overcome this limitation, we focused on the z-normalized gene expression data of 844 MM patients and use t-distributed Stochastic Neighbor Embedding (tsne) to construct an unbiased MM-specific transcriptomic landscape of 16,738 genes as shown in FIG. 11A. In turn, we used fuzzy c-means clustering to identify co-expressing gene clusters, or gene programs in MM (FIG. 11B). Using these unsupervised, MM-specific gene clusters, we carried out GSEA to examine expression patterns associated with resistance and sensitivity for a given drug, as shown in FIG. 11C for SELI. The pattern of enriched resistant and sensitive gene clusters on the transcriptomic map is referred to as a transcriptomic footprint, as it represents genes that not only correlate with ex vivo response but also co-express with each other in MM. These transcriptomic footprints are used to identify enriched transcription factors that bind to the genes implicated in resistance (red) and sensitivity (blue)(FIG. 11D). Similarly, FIG. 11E presents the enriched epigenetic alterations h3k27me3 and h3k27ac (key modifications driving MM progression) for resistance or sensitivity to various drugs tested ex vivo. FOXM1 is highly enriched for genes implicated in Volasertib (VOLA; PLK1 Inhibitor) sensitivity; where PLK1, a key regulator of cell cycle, is involved in a positive feedback loop with FOXM1 by phosphorylating FOXM1, which in turn regulates PLK1 activity. BI2536, on the other hand, a non-selective PLK inhibitor is not enriched for FOXM1, which suggests the specificity of the approach employed. Proteasome inhibitor (BTZ, CFZ, IXA) resistance is associated with RELA transcription factor, an NF-kb subunit, whose activity is associated with PI-resistance in MM and most likely regulated through ciap2 gene. BACH1 is exported from the nucleus by XPO1 (target of SELI) and recruits PRC2 to promote h3k27me3 modifications, which is further supported by the association of BACH1 and h3k27me3 being associated with resistance to SELI in FIGS. 11D and 11E, respectively. Six transcription factors (GABP, HOXC9, FOXP3, VDR, ETS1, and XRN2) are implicated in sensitivity for SELI (along with Crizotinib (CRIZ), MK2206, Ponatinib (PONA), Melphalan (MEL), and BI2536) and resistance for DARA (along with Motesanib and Alisertib). A detailed investigation of several transcription factors implicated in resistance and sensitivity to various drugs is presented in the table shown in FIG. 20.

### 3. Example 3: Ex Vivo Drug Sensitivity Prediction (Machine Learning Model)

This module involves training a regression tree-based machine learning model to estimate MM patient ex vivo drug sensitivity using gene expression data from RNA sequencing alone. The approach relies on co-expressing gene clusters implicated in resistance (shown in red) and sensitivity (shown in green) obtained from a biomarker discovery tool (described later in this section) to identify the genes that serve as input variables, whose expression is used to train a regression tree model using matched ex vivo drug sensitivity. FIG. 5 presents an overview of the approach, where the biomarker discovery tool was used to identify differentially enriched gene clusters that are not only differentially expressed but also co-express among each other for sensitivity to Selinexor. The regression tree model trained using this approach yields a correlation of 0.81 between model predicted area under the curve (AUC) and actual AUC. Further, the regression tree predicted aucs are validated clinically by comparing the model predicted aucs between responders and non-responders in a Selinexor-based regimen in the BOSTON clinical trial and shown to classify the two groups accurately with an AUC of receiver operator characteristic (ROC) curve equal to 0.7544.

### Selinexor Transcriptomic Footprints: Clinical Validation:

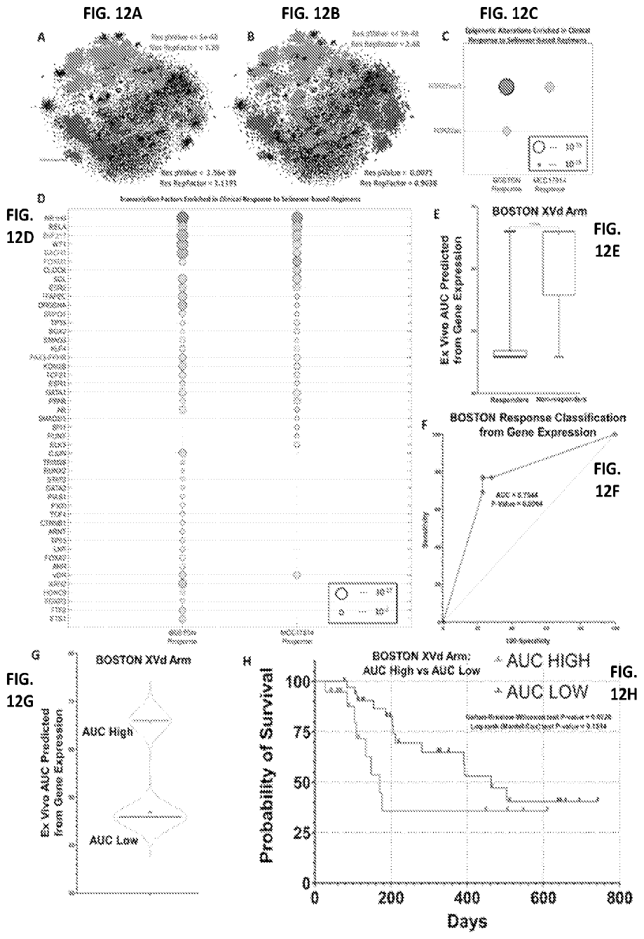

The transcriptomic footprints identified in FIG. 11C for ex vivo drug sensitivity (in blue) and resistance (in red) are validated by identifying co-expressing gene clusters enriched for resistance and sensitivity using GSEA by pairing RNA-seq data and clinical response stratification of patients classified as responders and non-responders as per IMWG response stratification in two independent clinical trials involving SELI-based regimens, BOSTON²⁵ and MCC17814. FIGS. 12A and 12B present gene sets enriched for clinical responders (blue) and non-responders (red) in each of the two trials. The significance of overlap of red genes associated with resistance in each of the two trials and those identified by ex vivo drug response to SELI are computed using a hypergeometric test with a p-value of less than 10⁻⁴⁸ for both BOSTON and MCC17814, and representation factors of 3.39 and 2.48, respectively. Similarly, the blue genes associated with ex vivo sensitivity to Selinexor are compared with those associated with clinical response in BOSTON and MCC17814 with a hypergeometric test p-value of 1.56×10⁻³⁹ and 0.0071 respectively and representation factors of 2.2291 and 0.9038. All comparisons show statistical significance beyond a threshold of 0.05 and a representation factor much greater than 1 (implying the overlap is greater than expected when the two sets of genes are chosen by random), except for the overlap of genes associated with ex vivo sensitivity and MCC17814 clinical response. This is likely due to a much smaller sample size and even fewer responders to a SELI-based regimen in MCC17814 involving a combination of SELI, DOX, and DEX. The BOSTON clinical trial, on the other hand, is a two-arm phase III clinical trial comparing the combination of SELI, BTZ, and DEX with a combination involving BTZ and DEX alone. It is important to note that the agreement between transcriptomic footprints identified using ex vivo and clinical response data validates the use of ex vivo response metrics to infer clinically relevant biomarkers. Moreover, it validates the approach used to identify these footprints. Finally, the transcriptomic footprints identified from the clinical response data alone typically involve contributions from other drugs in the regimen thereby preventing drug-specific mechanism discovery as shown in FIGS. 11D-11E. We also compare enriched epigenetic alterations and transcription factors for Selinexor clinical response (blue bubbles)/clinical resistance (red bubbles) to ex vivo drug sensitivity indicated by blue labels and resistance by red labels in FIGS. 12C and 12D.

We now investigate the possibility of using the transcriptomic footprint as a predictive biomarker of clinical response (depth of response, or progression free survival, PFS) to SELI-based regimens. To this end, we consider the 13 gene clusters (both red and blue) that form the SELI transcriptomic footprint as a gene signature. The median gene expression of each of these 13 clusters is used as an input to a regression tree model to compute a biomarker score for BOSTON trial patients to predict clinical response to a SELI-based treatment regimen. The regression tree model is trained using the matched ex vivo AUC (surrogates of clinical response as shown above) and median gene expression of the 13 clusters identified in the signature. We validate the model by estimating biomarker scores (predicted ex vivo AUC/surrogate for clinical response) for each patient treated with a SELI-based regimen in BOSTON using their RNA-seq data. In FIG. 12E, we present a box-and-whisker plot showing biomarker scores (predicted ex vivo AUC), based on the gene signature and gene expression data alone, on the y-axis for responders (blue) and non-responders (red) from the BOSTON clinical trial. By using the biomarker scores (predicted ex vivo AUC) as a classifier of clinical response we obtain a receiver-operator characteristic (ROC) curve with an AUC of 0.75 and a statistically significant p-value of 0.0064 demonstrating a high level of sensitivity and specificity of these molecular classifiers (FIG. 12F). FIG. 12G, on the other hand, uses the same biomarker scores (predicted ex vivo AUC) to divide the patients into those with a high score (red), associated with clinical resistance; and low score (blue), associated with ex vivo sensitivity. The probability of survival computed from PFS of BOSTON trial patients in the high score and low score groups is compared in FIG. 12H, where we see that the patients with a higher score have lower survival with a statistical significance of 0.022 as per the Gehan-Breslow Wilcoxon test. In summary, the transcriptomic footprints informed by ex vivo drug sensitivity data are reproduced accurately in a clinical setting (BOSTON and MCC17814), the footprint results in a gene signature that can be used as a surrogate for clinical response to be used as classifiers of clinical depth of response and progression free survival. This further validates the clinical relevance of the transcriptomic footprints.

### 4. Example 4: Novel Therapeutic Strategies Informed by Transcriptomic Footprints: Sequential Therapy of Selinexor and Daratumumab

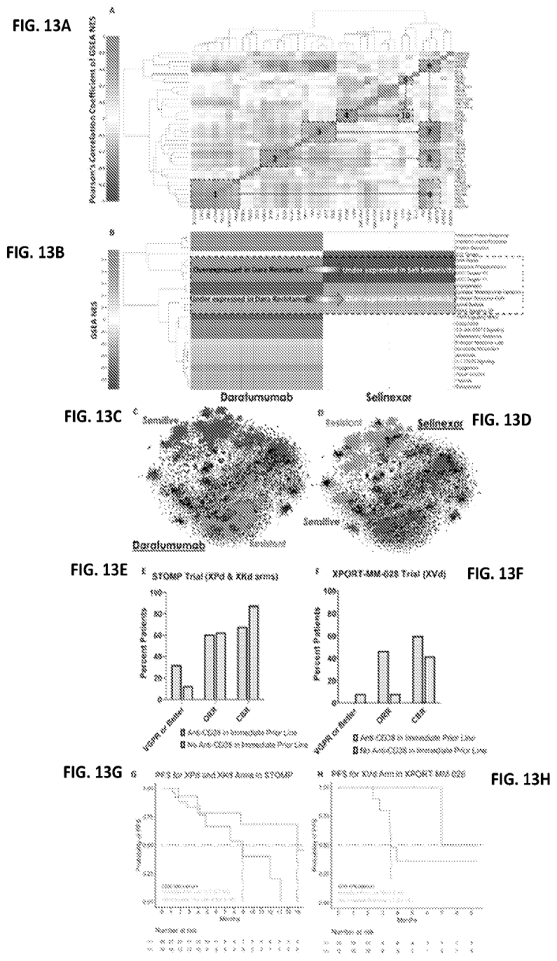

Next, we proposed that by examining the transcriptomic patterns associated with responses to anti-MM agents we would be able to identify pairs of drugs that could be sequenced for optimal therapeutic success (longer PFS). This train of thought followed that if the expression of genes associated with resistance to a specific drug and sensitivity to another, sequencing these therapies would increase the benefit of the second agent. Accordingly, we computed normalized enrichment scores (NES) for each gene cluster on the MM transcriptomic map shown in FIG. 11B for each agent tested ex vivo, where a positive NES corresponds to resistance to the drug and a negative NES is associated with sensitivity. We examined the similarity of transcriptomic footprints between drugs by estimating the correlation of NES scores of one drug with another. The resulting Pearson's correlation coefficients for every drug pair are presented as a clustergram in FIG. 13A. Here, we observed six clusters of drugs that correlate with each other indicated by dashed boxes and a number between 1 to 6. Importantly, we notice that drug clusters 1, 2, and 3 are negatively correlated with cluster 6, and cluster 4 is negatively correlated with cluster 5. Out of all the drug clusters, cluster 3 contained the most clinically relevant drugs and also has a negative correlation with cluster 6. From these two clusters, we focused on Selinexor (cluster 3) and Daratumumab (cluster 6) to investigate the benefit of sequential therapies. We have identified this anti-correlative profile involving some of the drug pairs with complementary association with resistance and sensitive phenotypes in FIGS. 11D and 11E based on enriched epigenetic alterations and transcription factors. In FIG. 13B, the heat map illustrates the NES of Cancer Hallmarks for ex vivo drug response to Selinexor and Daratumumab. The observed anti-correlate nature of NES led us to propose that Daratumumab and Selinexor are ideal candidates for sequential therapy, as the biology associated with resistance to one drug is implicated with sensitivity to the other (FIGS. 13C and 13D). We examined this hypothesis by comparing response rates of Selinexor-based regimens in STOMP (xpd and xkd arms) and XPORT-MM-028 (xvd) clinical trials between patients who were exposed to an anti-CD38 monoclonal antibody in the immediate prior line (yellow bars) versus those who didn't (blue bars). Consistent with our hypothesis, a higher VGPR rate in STOMP and a considerably higher ORR and CBR in XPORT-MM-028 for Selinexor-treated patients who were exposed to Daratumumab in an immediate prior line as shown in FIGS. 13E and 13F. Furthermore, we compared the PFS between these two groups in STOMP and XPORT-MM-028 clinical trials and, again, there was a statistically significant improvement in PFS for patients treated with Selinexor, who were exposed to an anti-CD38 monoclonal antibody in their immediate prior line. Collectively, these clinical data provide strong evidence for the rationale for sequencing Selinexor with Daratumumab as defined by our functional transcriptomics platform.

Despite significant gains in our treatment armamentarium, MM remains an all but incurable cancer of bone marrow resident plasma cells. We anticipate that these gains could be further extended via the identification of optimal therapeutic intervention using predictive biomarkers. While diagnostic and prognostic biomarkers are integrated into clinical utilization in MM, predictive biomarkers remain notoriously absent from clinical use. Here, we utilize a functional transcriptomics—ex vivo drug screening as patient avatars (surrogates for clinical response) and paired molecular data—to identify critical MM biology and predictive biomarkers and validated them in independent clinical trials.

Patient-derived MM cells were co-cultured in an ex vivo reconstruction of the tumor microenvironment in a multi-well plate and tested with several drugs to estimate patient-drug-specific estimates, leading to the world's largest drug sensitivity database in MM featuring 400 patients tested with 38 drugs. We paired ex vivo drug sensitivity estimates with clinical disease state, cytogenetic abnormalities from FISH, and driver mutations from WES to identify genomic biomarkers of ex vivo drug sensitivity in MM. Owing to a very low frequency of mutations in MM, we have a very sparse genomic landscape. For this reason, we rely on z-normalized gene expression data to construct an MM transcriptomic landscape that results in gene clusters that co-express in MM, which are subjected to GSEA using paired ex vivo drug sensitivity data and RNA-seq data to identify gene clusters that correlate with resistance or sensitivity, and co-express in MM patients. This resulted in identifying drug-specific transcriptomic footprint for Selinexor, which is reproduced using clinical response data from BOSTON and MCC17814. The gene signature obtained from the transcriptomic footprint was used to train a regression tree model to predict ex vivo drug sensitivity from gene expression data alone. The model accurately predicts aucs, and the predicted aucs can be used as effective classifiers of therapy response and progression free survival. The transcriptomic footprints for each of the 38 drugs are correlated with each other to identify pairs of drugs with anti-correlative transcriptomic profiles. Selinexor and Daratumumab were chosen as ideal candidates for sequential therapy due to their anti-correlative transcriptomic profile that suggests that the biology associated with resistance to one drug is associated with sensitivity to the other. This novel therapeutic strategy was validated in two clinical trials, STOMP and XPORT-MM-028, where patients treated with a Selinexor-based regimen who received an anti-CD38 monoclonal antibody in an immediate prior line had deeper response and longer PFS.

To discover the underlying mechanism driving clinical benefit due to sequential therapy of Selinexor and Daratumumab, we rely on the transcriptomic footprints showcased in FIGS. 13C and 13D. We consider the gene clusters over expressed in patients resistant to Selinexor and note that these clusters are also associated with h3k27me3 histone modifications in MM samples inferred from single-cell ATAC sequencing data, and confirmed by the enrichment analysis presented in FIG. 12E. These gene clusters are also enriched for immune/microenvironment-mediated mechanisms like IL2 STAT3 signaling, inflammatory response, IL6 JAK STAT3 signaling, and complement in MM patient samples. These mechanisms are expected to be involved in Daratumumab mechanism of action. Hence, cancer cells exposed to Daratumumab and acquire resistance to it would have lower expression of the genes in these clusters as indicated by FIG. 13C. Selinexor inhibits XPO1 expression, which is responsible for exporting BACH1 from the nucleus to recruit PRC2 and promoting h3k27me3 modifications. XPO1 inhibition hinders BACH1-mediated h3k27me3 modifications, which lead to greater chromatin accessibility and sensitivity to Selinexor.

In summary, we relied on pre-clinical ex vivo response and transcriptomic data to identify a novel therapeutic strategy in MM, that is shown to be beneficial in a clinical setting. The conventional model for developing novel therapeutic strategies typically involves relying on clinical trials based on pre-clinical data obtained from hypothesis-driven studies. The probability of success (POS) for oncology drugs in a phase III clinical trial is estimated to be 35.5% in comparison to an estimated POS of 63.6% for all other therapeutic groups combined (endocrinology, cardiovascular, vaccines, etc.). Hence, there is a dire need to improve the odds of success of oncology drugs investigated in a phase III clinical trial by identifying novel therapeutic strategies informed by data-driven approaches as described in this article.

### 5. Example 5: Gene Regulatory Network Model

Gene Set Enrichment Analysis (GSEA) of ex vivo drug response, and matched gene expression of MM patients, identified differentially expressed gene (DEG) clusters. Upstream regulatory proteins (RPs) that selectively target DEG clusters were found using publicly available databases (ENCODE/ChEA) of genome-wide chip-X experiments. Kinases targeting these RPs were identified from PSP, generating a cascading network of DEGs, RPs, and kinases described by a model of ordinary differential equations for transcription, translation, and post-translational effects.

The proposed method identified 197 DEGs in MM patients resistant to proteasome inhibitors (PIs; front-line therapy in MM), 13 upstream RPs, and 45 kinases (17 targetable using kinase inhibitors). The proposed model was trained with expression data of upstream RPs and kinases from 430 randomly selected patients and validated in a 414 patient cohort. 177/197 DEGs showed a strong linear (Pearson's) correlation in the validation cohort as shown in Table 1. CDK inhibitors Seliciclib and Dinaciclib were predicted to effect DEGs with statistical significance (0.05) in 87.5% and 84.9% patients using a paired t-test. Dinaciclib's role in PI-resistant patients was functionally validated ex vivo, showing synergy with PIs.

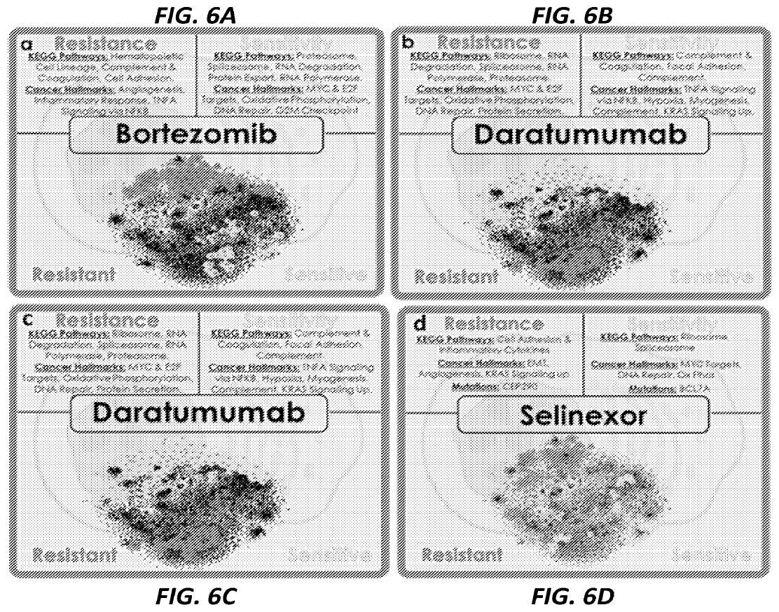

The Biomarker Discovery Tool was implemented on three drugs; bortezomib (a proteasome inhibitor), selinexor (a nuclear-export inhibitor) and daratumumab (a monoclonal antibody). FIG. 8a presents the tsne map showing gene clusters enriched for resistance in red and sensitivity in green to bortezomib. KEGG pathways and cancer hallmarks enriched for resistance and sensitivity are also listed. FIGS. 6b and 6c show the same for daratumumab, and 6d for Selinexor; where we note complementary resistance profiles on the tsne map. We interface this biomarker discovery tool with a gene regulatory network (GRN) model that identifies candidate kinase inhibitors that can overcome resistance to SOC drugs in MM.

We programmatically construct GRNs that connect druggable kinases to each gene cluster of the MM transcriptomic map. These two layers of the network are connected by a layer of transcription factors that act as putative master regulators in driving mechanisms of resistance, and are phosphorylated by several druggable kinases, thereby providing a mechanism to pharmacologically reach the gene cluster. As genome-wide regulatory networks haven't yet been developed with reliable accuracy⁹, we utilize several publicly available databases to infer connections between the gene cluster, transcription factors (TFs), and kinases. ENCODE and ChEA are two such sources providing extensive data linking transcription factors to genes from genome-wide chip-X experiments. This information is used to identify upstream transcription factors for each cluster by conducting a hypergeometric t-test for the representation of each gene set from the list of genes bound by a given transcription factor. We use a threshold of p-value less than 0.05 and a representation factor greater than one. Each of these upstream transcription factors maybe phosphorylated by several kinases, which is inferred from publicly available proteomic databases phosphositeplus and phosphopoint. This leads to a hierarchical network that forms a cascade of target genes, transcription factors, and kinases as shown in FIG. 7c. While most GRNs only inform associations between nodes and directionality of the edges, here we propose a novel implementation to simulate the effect of treatment in patient-specific MM cells, by establishing functional relationships between the nodes, where each node is either a target gene (from a gene cluster), or transcription factor, or a kinase. Mechanistically, the process of transcription links transcription factors to target genes, post-translational modification links kinases to transcription factors, and translation estimates the amount of activated protein from gene expression of transcription factors and kinases. By modeling these three processes from first principles using a biophysical model (in 7d-7f), and assuming steady state we obtain a functional relationship between gene expression of transcription factors and kinases, and target genes as shown in FIG. 7g. Moreover, the model includes patient-specific rate constants for ribosome (Krb), protein degradation (Kpd), RNA synthesis (KRNA), and RNA degradation (Krd); which are all known KEGG pathways that have ssgsea (single-sample gene set enrichment analysis) scores that represent enrichment of each of these pathways in each patient. We model these patient-specific rate constants as a function of that patient's ssgsea scores, where the rate constant can vary between a fixed range and the precise value for a given patient is determined by the enrichment score. The training cohort of 844 MM patients is equally divided into GRN model training and validation cohorts, where the model parameters for each target gene within a cluster are estimated by fitting the proposed biophysical model to the target gene, upstream tfs, and kinase expression of patients in the training cohort (as shown in FIG. 8a). The model parameters estimated from the training cohort are used to predict the expression of target genes for the validation cohort and are correlated with the actual gene expression (as shown in FIG. 8b). Similarly, model parameters for all genes in all clusters are estimated and Pearson's correlation coefficients are computed for the validation cohort as shown in Table 1 for an example gene cluster (implicated in cell cycle regulation) with 197 genes.

TABLE 1

| DEG | R_training | R_validation | Regression_slope |
|---|---|---|---|
| CDK1 | 0.970 | 0.971 | 0.816 |
| PBK | 0.969 | 0.963 | 0.746 |
| TTK | 0.955 | 0.952 | 0.850 |
| PLK1 | 0.956 | 0.943 | 0.974 |
| CCNA2 | 0.969 | 0.934 | 0.932 |
| AURKA | 0.939 | 0.929 | 1.007 |
| CCNB2 | 0.895 | 0.925 | 0.890 |
| ORC1 | 0.950 | 0.920 | 0.995 |
| CDKN3 | 0.903 | 0.920 | 0.972 |
| UBE2C | 0.935 | 0.920 | 0.939 |
| CCNB1 | 0.939 | 0.919 | 0.941 |
| ESCO2 | 0.906 | 0.917 | 0.982 |
| DLGAP5 | 0.951 | 0.910 | 1.084 |
| NCAPH | 0.896 | 0.906 | 0.902 |
| CDC20 | 0.959 | 0.903 | 1.120 |
| TOP2A | 0.942 | 0.898 | 0.937 |
| KIF2C | 0.905 | 0.898 | 0.848 |
| DEPDC1 | 0.910 | 0.892 | 1.164 |
| NCAPG | 0.923 | 0.889 | 0.918 |
| CDCA2 | 0.903 | 0.882 | 0.759 |
| BIRC5 | 0.912 | 0.871 | 1.025 |
| CENPA | 0.913 | 0.870 | 0.829 |
| NUSAP1 | 0.860 | 0.868 | 0.838 |
| FAM83D | 0.911 | 0.864 | 1.066 |
| CDC25A | 0.878 | 0.863 | 0.840 |
| FAM72B | 0.867 | 0.861 | 0.967 |
| TRIP13 | 0.905 | 0.861 | 0.953 |
| KIF20A | 0.917 | 0.861 | 0.884 |
| SKA1 | 0.902 | 0.860 | 0.946 |
| EXO1 | 0.918 | 0.860 | 0.933 |
| HJURP | 0.895 | 0.858 | 0.996 |
| NEK2 | 0.920 | 0.857 | 0.924 |
| PRC1 | 0.863 | 0.857 | 1.037 |
| NUF2 | 0.896 | 0.855 | 0.987 |
| DEPDC1B | 0.868 | 0.855 | 0.761 |
| KIF11 | 0.923 | 0.854 | 0.882 |
| CDCA8 | 0.916 | 0.851 | 0.926 |
| KIF23 | 0.887 | 0.851 | 0.907 |
| KIF4A | 0.932 | 0.850 | 0.926 |
| KIFC1 | 0.826 | 0.850 | 0.780 |
| ORC6 | 0.870 | 0.849 | 0.911 |
| PTTG1 | 0.894 | 0.848 | 0.961 |
| BUB1 | 0.918 | 0.846 | 0.888 |
| MCM7 | 0.895 | 0.845 | 0.879 |
| DSCC1 | 0.899 | 0.845 | 0.980 |
| KIF22 | 0.869 | 0.844 | 0.957 |
| MCM4 | 0.899 | 0.835 | 1.112 |
| RAD51 | 0.894 | 0.835 | 0.964 |
| CIT | 0.852 | 0.834 | 0.932 |
| ARHGAP11A | 0.890 | 0.834 | 0.943 |
| POLE2 | 0.868 | 0.834 | 0.833 |
| HMMR | 0.893 | 0.833 | 0.889 |
| CDCA3 | 0.878 | 0.833 | 0.805 |
| RACGAP1 | 0.864 | 0.829 | 0.967 |
| UBE2T | 0.920 | 0.826 | 0.827 |
| EZH2 | 0.836 | 0.824 | 0.905 |
| TPX2 | 0.898 | 0.823 | 0.919 |
| PARPBP | 0.867 | 0.821 | 0.929 |
| CCNF | 0.921 | 0.821 | 0.986 |
| CENPE | 0.864 | 0.819 | 0.876 |
| SKA3 | 0.887 | 0.816 | 0.869 |
| PLK4 | 0.902 | 0.815 | 0.861 |
| ZWINT | 0.877 | 0.814 | 0.927 |
| MCM2 | 0.876 | 0.814 | 1.052 |
| RRM2 | 0.886 | 0.811 | 0.952 |
| MCM5 | 0.843 | 0.809 | 0.876 |
| E2F8 | 0.905 | 0.809 | 0.807 |
| NDC80 | 0.879 | 0.805 | 0.809 |
| DTL | 0.912 | 0.805 | 0.822 |
| CDC45 | 0.871 | 0.805 | 0.924 |
| CDCA4 | 0.904 | 0.804 | 0.831 |
| ACOT7 | 0.864 | 0.803 | 1.032 |
| DIAPH3 | 0.876 | 0.801 | 0.827 |
| ECT2 | 0.874 | 0.798 | 0.903 |
| FOXM1 | 0.805 | 0.798 | 1.216 |
| POC1A | 0.853 | 0.797 | 0.883 |
| INCENP | 0.828 | 0.797 | 0.813 |
| CDCA5 | 0.769 | 0.796 | 0.705 |
| GAS2L3 | 0.835 | 0.796 | 0.924 |
| ATAD5 | 0.876 | 0.793 | 0.881 |
| CDT1 | 0.892 | 0.792 | 0.983 |
| MKI67 | 0.807 | 0.792 | 0.996 |
| UBE2S | 0.790 | 0.791 | 0.877 |
| CDC6 | 0.871 | 0.788 | 0.878 |
| FANCI | 0.866 | 0.787 | 0.944 |
| STIL | 0.858 | 0.786 | 0.781 |
| SPC25 | 0.857 | 0.784 | 0.912 |
| WDHD1 | 0.875 | 0.781 | 0.879 |
| SHCBP1 | 0.888 | 0.781 | 0.876 |
| CENPF | 0.881 | 0.778 | 1.048 |
| ANLN | 0.877 | 0.777 | 1.060 |
| XRCC3 | 0.863 | 0.776 | 0.812 |
| CEP55 | 0.842 | 0.776 | 0.756 |
| TK1 | 0.825 | 0.774 | 0.862 |
| MELK | 0.891 | 0.774 | 0.949 |
| FAM111B | 0.768 | 0.773 | 0.847 |
| TYMS | 0.867 | 0.770 | 0.957 |
| CDC25C | 0.832 | 0.767 | 0.853 |
| ESPL1 | 0.824 | 0.765 | 0.899 |
| GTSE1 | 0.801 | 0.764 | 1.002 |
| SPAG5 | 0.887 | 0.761 | 0.824 |
| MCM10 | 0.868 | 0.759 | 0.833 |
| BRCA1 | 0.779 | 0.758 | 0.851 |
| WDR76 | 0.847 | 0.758 | 0.952 |
| ASPM | 0.853 | 0.757 | 0.861 |
| MCM3 | 0.768 | 0.751 | 0.895 |
| KIF18A | 0.886 | 0.748 | 0.832 |
| TROAP | 0.824 | 0.747 | 0.825 |
| TICRR | 0.817 | 0.747 | 0.871 |
| CENPH | 0.850 | 0.747 | 0.847 |
| TONSL | 0.851 | 0.746 | 0.839 |
| MYBL2 | 0.880 | 0.746 | 0.899 |
| TRAIP | 0.799 | 0.746 | 0.770 |
| BRI3BP | 0.787 | 0.746 | 0.789 |
| POLQ | 0.891 | 0.744 | 0.836 |
| KIF14 | 0.900 | 0.740 | 0.870 |
| CCDC15 | 0.846 | 0.738 | 0.874 |
| NCAPG2 | 0.834 | 0.731 | 0.942 |
| BUB1B | 0.868 | 0.731 | 1.054 |
| EME1 | 0.734 | 0.726 | 0.915 |
| AURKB | 0.899 | 0.725 | 0.944 |
| TIMELESS | 0.808 | 0.724 | 0.826 |
| PKMYT1 | 0.888 | 0.724 | 0.882 |
| ZWILCH | 0.789 | 0.723 | 0.720 |
| CENPI | 0.788 | 0.722 | 0.807 |
| KIF15 | 0.903 | 0.721 | 0.752 |
| KIF18B | 0.904 | 0.720 | 1.002 |
| NCAPD3 | 0.792 | 0.719 | 0.817 |
| PHF19 | 0.825 | 0.715 | 0.818 |
| FAM72A | 0.792 | 0.711 | 0.928 |
| BRIP1 | 0.779 | 0.707 | 0.841 |
| HELLS | 0.873 | 0.705 | 0.905 |
| RAD54L | 0.753 | 0.705 | 0.909 |
| UHRF1 | 0.859 | 0.703 | 1.075 |
| DNMT3B | 0.747 | 0.699 | 0.990 |
| KIF24 | 0.816 | 0.698 | 0.735 |
| KREMEN2 | 0.909 | 0.695 | 0.903 |
| GINS2 | 0.597 | 0.695 | 1.427 |
| CLSPN | 0.808 | 0.694 | 0.954 |
| OIP5 | 0.821 | 0.694 | 1.321 |
| PRR11 | 0.886 | 0.690 | 0.946 |
| STMN1 | 0.836 | 0.686 | 0.704 |
| RECQL4 | 0.708 | 0.686 | 0.865 |
| CENPK | 0.854 | 0.683 | 0.872 |
| WDR62 | 0.773 | 0.671 | 0.988 |
| MIS18A | 0.668 | 0.667 | 0.747 |
| E2F2 | 0.823 | 0.666 | 0.864 |
| XRCC2 | 0.828 | 0.666 | 0.964 |
| RAC3 | 0.636 | 0.664 | 0.905 |
| PSMC3IP | 0.795 | 0.664 | 0.791 |
| MND1 | 0.873 | 0.658 | 0.824 |
| CKAP2L | 0.882 | 0.655 | 0.864 |
| SPA17 | 0.823 | 0.652 | 0.799 |
| CENPM | 0.807 | 0.651 | 1.015 |
| CHAF1B | 0.814 | 0.641 | 0.788 |
| SPC24 | 0.636 | 0.638 | 1.252 |
| NEIL3 | 0.814 | 0.626 | 0.987 |
| ITPRIPL1 | 0.703 | 0.621 | 0.879 |
| ASF1B | 0.617 | 0.606 | 0.604 |
| E2F1 | 0.676 | 0.602 | 0.993 |
| CCDC150 | 0.637 | 0.601 | 1.101 |
| SCML2 | 0.758 | 0.601 | 0.928 |
| SPDL1 | 0.625 | 0.600 | 0.736 |
| RIBC2 | 0.666 | 0.594 | 0.813 |
| ARHGAP11B | 0.734 | 0.594 | 1.236 |
| WDR34 | 0.725 | 0.565 | 0.830 |
| SAPCD2 | 0.738 | 0.565 | 0.861 |
| GINS4 | 0.591 | 0.560 | 0.778 |
| GINS1 | 0.540 | 0.560 | 1.442 |
| ARHGEF39 | 0.569 | 0.559 | 0.848 |
| FBXO43 | 0.433 | 0.546 | 1.941 |
| IQGAP3 | 0.841 | 0.540 | 0.827 |
| TCF19 | 0.636 | 0.530 | 0.688 |
| KANK2 | 0.760 | 0.525 | 0.981 |
| ZNF367 | 0.796 | 0.511 | 0.744 |
| DMC1 | 0.628 | 0.510 | 0.668 |
| SMC1B | 0.727 | 0.507 | 1.059 |
| ALDH4A1 | 0.776 | 0.488 | 0.695 |
| MNX1 | 0.196 | 0.445 | 5.681 |
| APOBEC3B | 0.695 | 0.414 | 0.933 |
| ERCC6L | 0.626 | 0.411 | 4.145 |
| RTKN2 | 0.713 | 0.396 | 0.679 |
| MAR-3 | 0.394 | 0.392 | 2.626 |
| E2F7 | 0.458 | 0.387 | 2.045 |
| ELOVL6 | 0.908 | 0.379 | 0.828 |
| DPF1 | 0.662 | 0.359 | 0.775 |
| DRP2 | 0.656 | 0.346 | 0.575 |
| NOSTRIN | 0.381 | 0.330 | 1.568 |
| UMODL1 | 0.644 | 0.321 | 0.494 |
| RDM1 | 0.557 | 0.317 | 0.585 |
| INTU | 0.584 | 0.306 | 0.620 |
| GALNTL6 | 0.543 | 0.297 | 0.687 |
| ACBD7 | 0.416 | 0.251 | 3.304 |
| MECOM | 0.445 | 0.248 | 1.029 |
| CCDC155 | 0.311 | 0.199 | 0.653 |
| ACRV1 | 0.576 | 0.186 | 0.659 |
| MYBPC2 | 0.364 | 0.183 | 1.914 |

The genes with a correlation greater than 0.5 are highlighted in red in FIG. 8c on the MM transcriptomic map, while the genes with a significant chromatin variability (normalized standard deviation of chromatin accessibility from 10 patient samples) are highlighted in yellow in FIG. 8d. A juxtaposition of the two plots reveals a near-perfect complementary overlap, implying the genes with poor model predictions have high variability in chromatin accessibility. Most of the genes can be modeled using gene expression alone (which are implicated in resistance mechanisms of several drugs), however, the remaining require both gene expression and chromatin accessibility. We have collated a comprehensive list of over 250 kinase inhibitors and their respective primary targets, which is obtained from vendors (e.g. Selleckchem) and literature. The effect of each kinase inhibitor can be simulated by silencing the corresponding kinases in the network listed as primary targets and estimating the target gene expression for all the target genes. The effect of the kinase inhibitor can be quantified using a normalized enrichment score (NES) for each cluster using a running-sum statistic of the log¬2 (fold-change) of gene expression in a ranked-list of genes from highest log 2 (fold-change) to the least. Next, we can rank the clusters based on their NES to determine specificity of each kinase inhibitor's effect. The kinase inhibitors that selectively influence the expression of target genes (resistance cluster) can be identified using the NES rank of the target cluster in each patient. The highly ranked kinase inhibitors across all patients, found using a consensus or mean of NES ranks, are considered candidate kinase inhibitors that can help overcome, or delay resistance to an SOC drug.

### 6. Example 6: Identifying Resensitizers to Overcome, or Delay SOC Drug Resistance

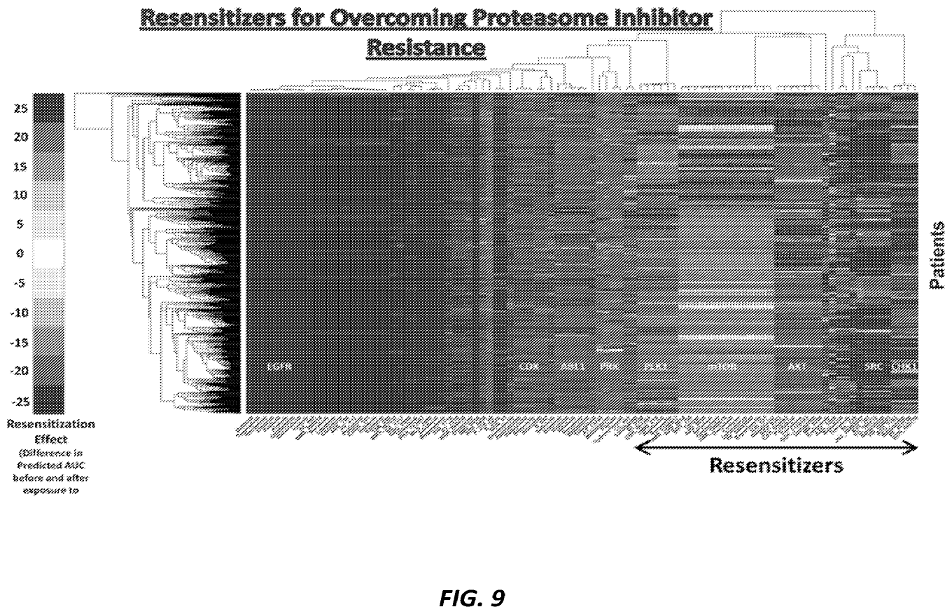

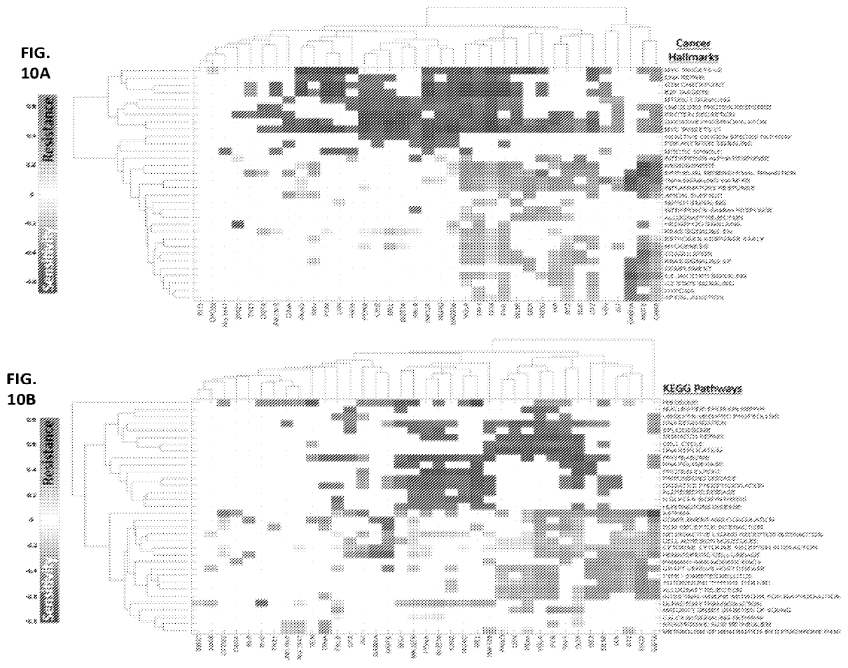

The modified gene expression profile computed by the GRN model is obtained by simulating the effect of a targeted therapy and is passed into the machine learning (regression tree, or neural network) model to predict the modified ex vivo drug sensitivity of the patient. FIG. 9 shows a clustergram of resensitization (difference in predicted drug response using modified gene expression and the patient's actual gene expression profiles—benefit of resensitization) for each candidate resenstizer (column) targeting proteasome inhibitor resistance across all patients (rows). A blue cell in the heatmap indicates that the candidate resensitizer (column) greatly benefits the patient (row), while a red cell implies poor resensitization effect.

### 7. Example 7: Characterization of Synergistic Selinexor Combinations with Dexamethasone, Pomalidomide, Elotuzumab, and Daratumumab in Primary MM cells

### Methods

We established a platform to perform parallel RNN/exome sequencing and ex vivo drug sensitivity assessment on CD138+ cells from MM patient bone marrow aspirates. At the time of this analysis, 844 different samples with clinical, WES and RNA sequencing data were treated ex vivo with the following agents: SELI (n=75), DEX (192), pomalidomide (POM, 268), elotuzumab (ELO, 21), daratumumab (DARA, 117), sell+DEX (22), sell+POM (20), sell+ELO (21), sell+DARA (27). Cells were cultured with autologous macrophages, stroma, collagen matrix and patient-derived serum. Cell death (LD₅₀ and AUC) was assessed through digital image analysis. Sequencing was performed through ORIEN/AVATAR. Links between non-synonymous mutations in coding genes and cell death were calculated using T-tests with multiple test correction.

### Results

Our analysis identified SELI+DEX (number of samples=60, p<1 E-9), SELI+POM (57, p<0.001) and SELI+ELO (55, p<0.01) as the most synergistic combinations (BLISS model). SELI+DARA showed synergy in 23 out of 50 samples tested. Notably, both direct drug toxicity and phagocytosis were observed. Rnaseq found gene expression associations with drug resistance/response. In turn, gene set enrichment analysis (GSEA) showed that SELI resistance was associated with expression of cell adhesion, inflammatory cytokines, and EMT pathways, while the MYC targets were associated with SELI sensitivity. SELI+ELO resistance was associated with expression of hedgehog signaling pathway, while expression of ribosomal subunits was associated with sensitivity. SELI+POM resistance was linked with lysosome and cell adhesion molecules, while sensitivity was tied to ribosome, spliceosome and RNA polymerase. GSEA also identified G2M, MTORC7, MYC targets, E2F and glycolysis as biomarkers for the sell+DARA synergistic subgroup. WES also identified mutations associated with SELI sensitivity. Mutation of BCL7A, a protein involved in chromatin remodeling, was associated with sensitivity, and mutation of CEP290, which encodes a microtubule binding protein, was associated with resistance (p<0.05). Both BCL7A and CEP290 contain predicted nuclear export sequences, suggesting they are XPO1 cargoes.

### CONCLUSIONS

We observed ex vivo synergy between SELI and DEX, POM, ELO and DARA, and identified expression signatures and mutations associated with response to these agents.

### 8. Example 8: Characterization of Synergistic Selinexor Combinations with Dexamethasone, Pomalidomide, Elotuxumab, and Daratumumab in Primary MM Cells

In this example, we characterize synergistic combinations with Selinexor in multiple myeloma. Selinexor is an oral Selective Inhibitor of Nuclear Export, which is an FDA approved drug to treat multiple myeloma, prescribed in combination with dexamethasone for patients who have received at least four lines of therapy, and in combination with bortezomib and dexamethasone for patients who received one to three prior lines of therapy. We study the nature of synergism in Selinexor combinations with dexamethasone, pomalidomide, elotuzumab, and daratumumab by identifying molecular biomarkers using a computational framework that relies on matched ex vivo drug response, RNA-sequencing, and whole exome sequencing data of primary myeloma cells.

We estimate ex vivo drug sensitivity for each myeloma patient across several single agents and combinations using an assay, where primary myeloma cells are co-cultured in an ex vivo reconstruction of the tumor microenvironent and are subjected to live imaging for up to six days. The percent viability measures across time are used to compute LD₅₀ and area under the curve for each patient-drug pair.

This drug sensitivity data is matched with the patient's RNA-seq and whole exome sequencing data.

These serve as inputs to the computational framework, where we obtain a myeloma-specific transcriptomic map via a two-dimensional embedding of over 16,000 genes from RNA-seq data of 844 patients using t-SNE followed by fuzzy c-means clustering to identify co-expressing gene clusters.

For each combination, we quantify the improvement in response over a theoretically computed additive response from the single agents, which serves as a measure of synergy. We use GSEA to identify differentially expressed gene clusters based on intensity of synergy and antagonism and identify gene sets from KEGG and Hallmarks that are enriched for these conditions, along with mutations from whole exome sequencing.

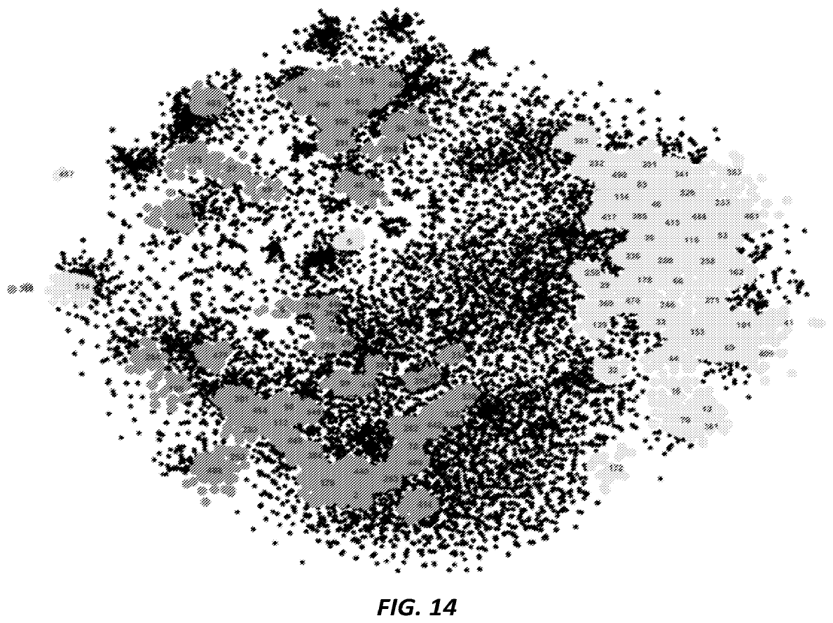

At the center of it all lies Selinexor. We used our computational framework to identify differentially expressed gene clusters based on resistance highlighted in red and sensitivity highlighted in green (FIG. 14). We also identified KEGG pathways, Cancer Hallmarks and mutations that are enriched for intensity of resistance and sensitivity to Selinexor.

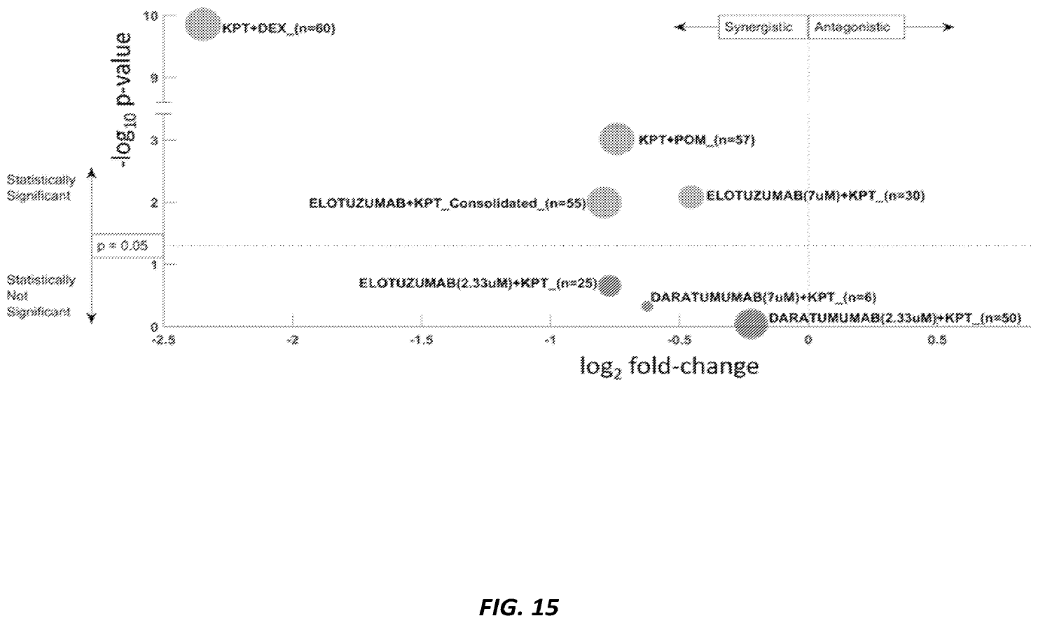

However, our primary goal is to characterize synergistic combinations with Selinexor. The volcano plot shows four combinations explored in this study, where the extent of synergy in terms of the log 2 fold-change over an additive response is shown along the x-axis and the likelihood of synergy within the patient cohort as −log 10 p-value is shown along the y-axis (FIG. 15). Combinations featured to the right of the plot are more synergistic, while the ones further up show synergy in more patients. The combination of Selinexor and dexamethasone is the most synergistic pair, followed by Selinexor and pomalidomide, and Selinexor and elotuzumab. The combination with daratumumab, although trends towards synergy is not statistically significant in our cohort.

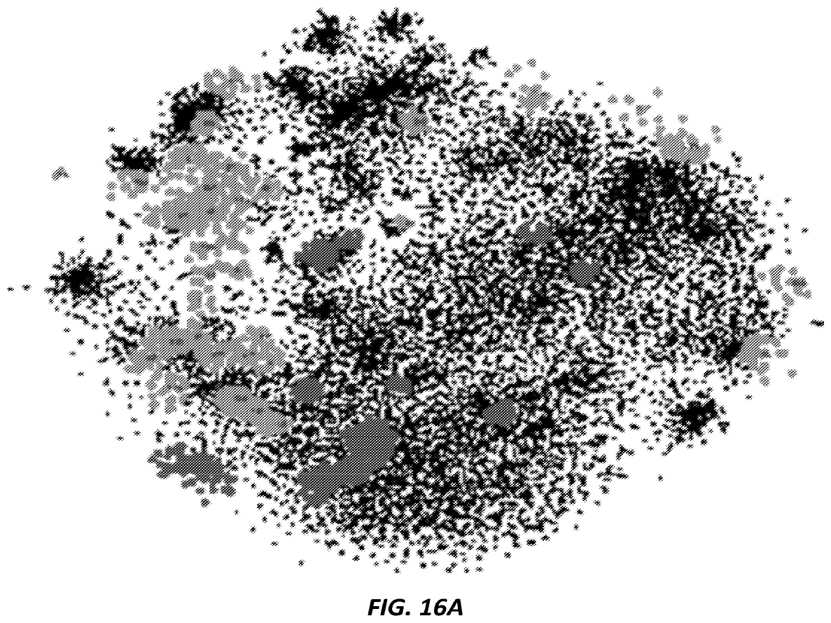

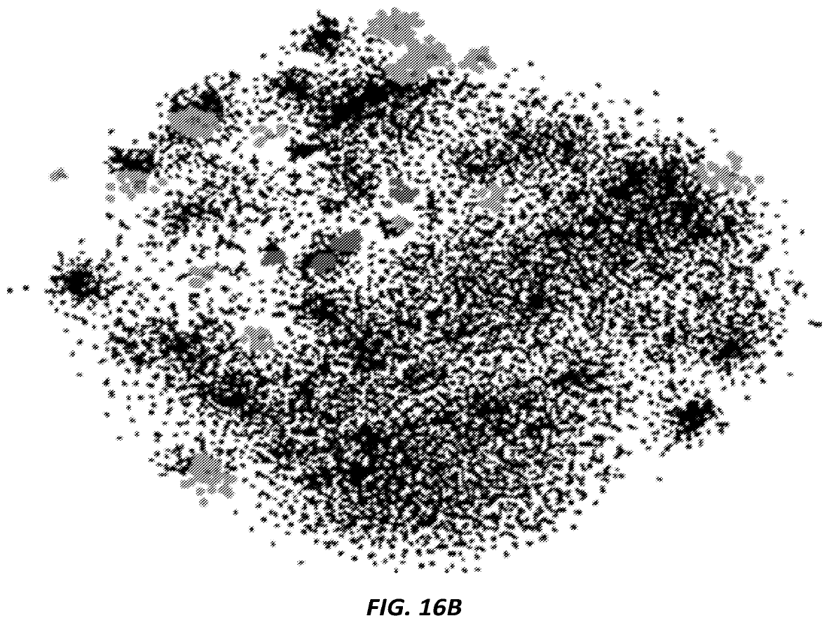

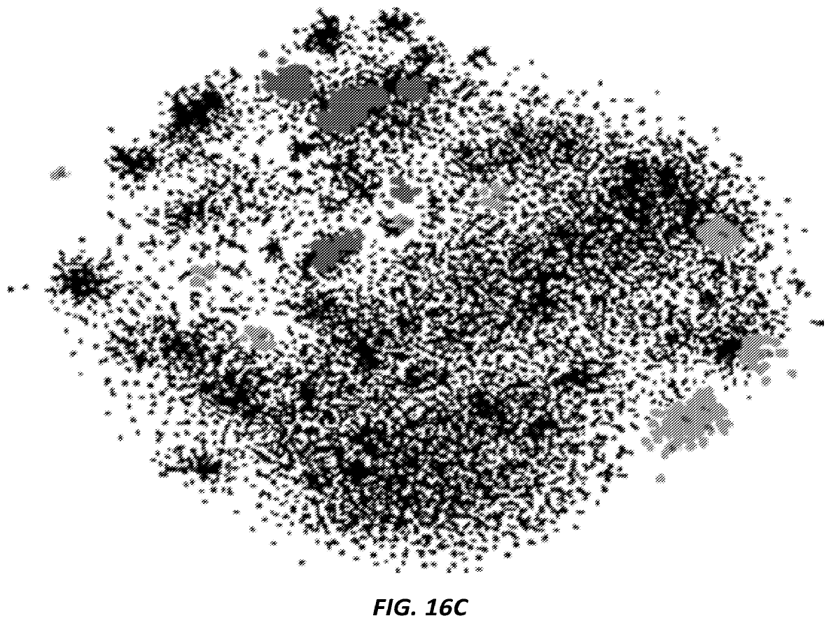

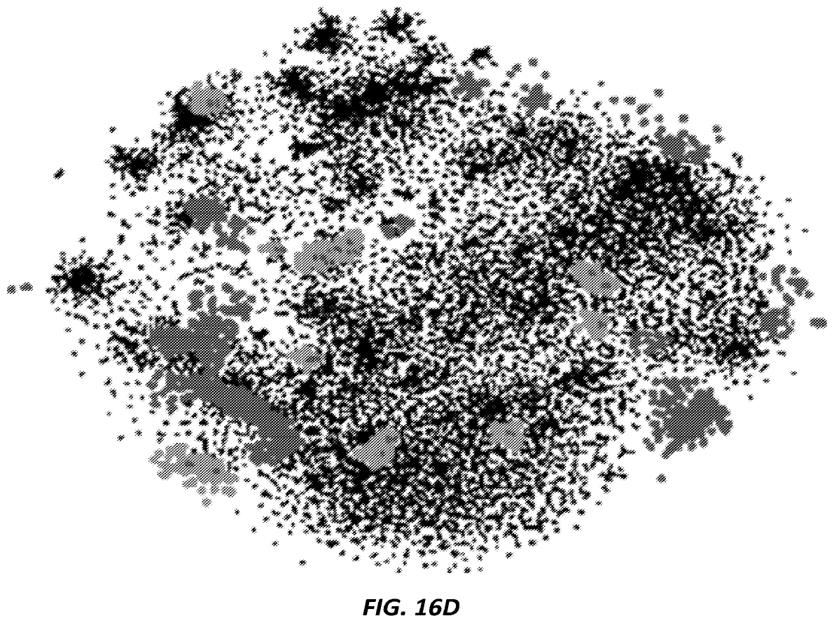

The transcriptomic maps highlighting clusters based on intensity of synergism (red) and antagonism (blue) for each of the four combinations are shown along with clusters enriched for intensity of resistance and sensitivity with Selinexor alone (FIGS. 16A-16D).

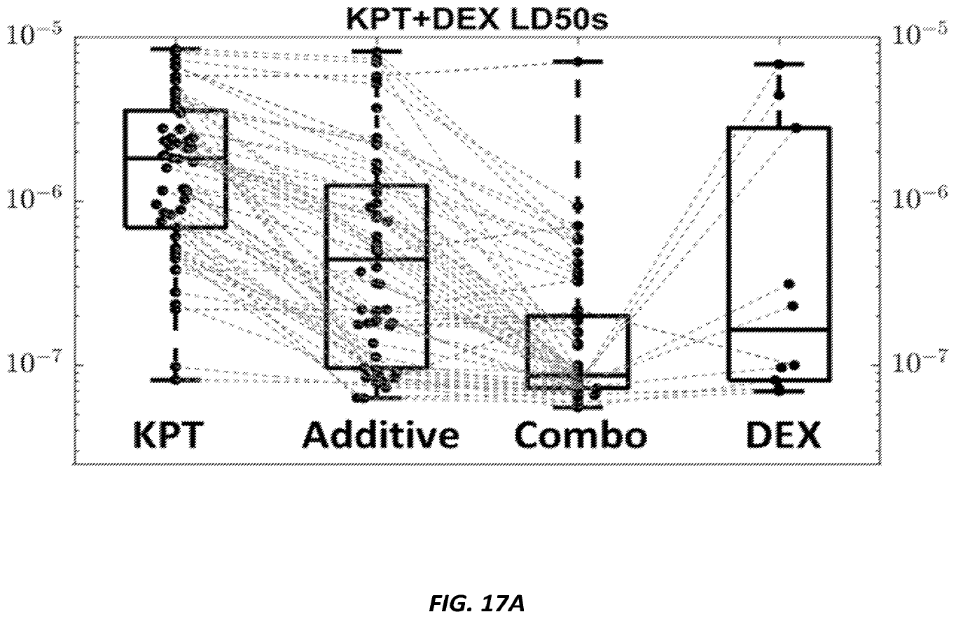

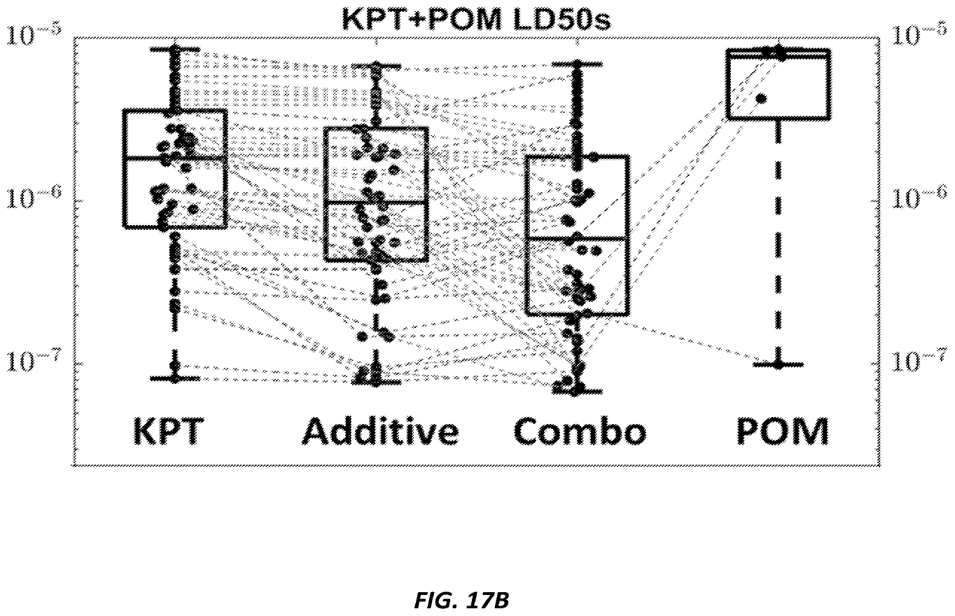

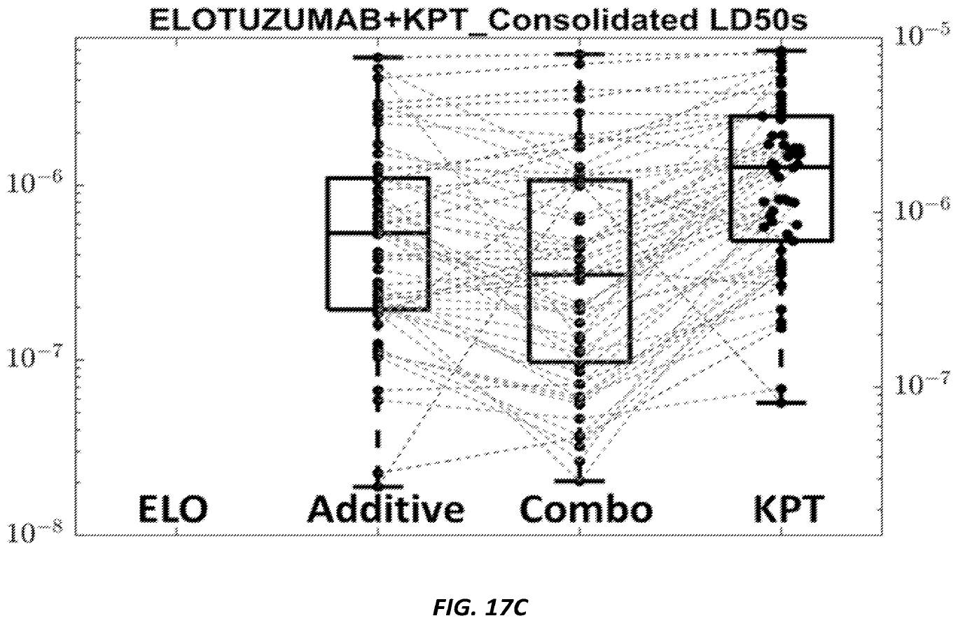

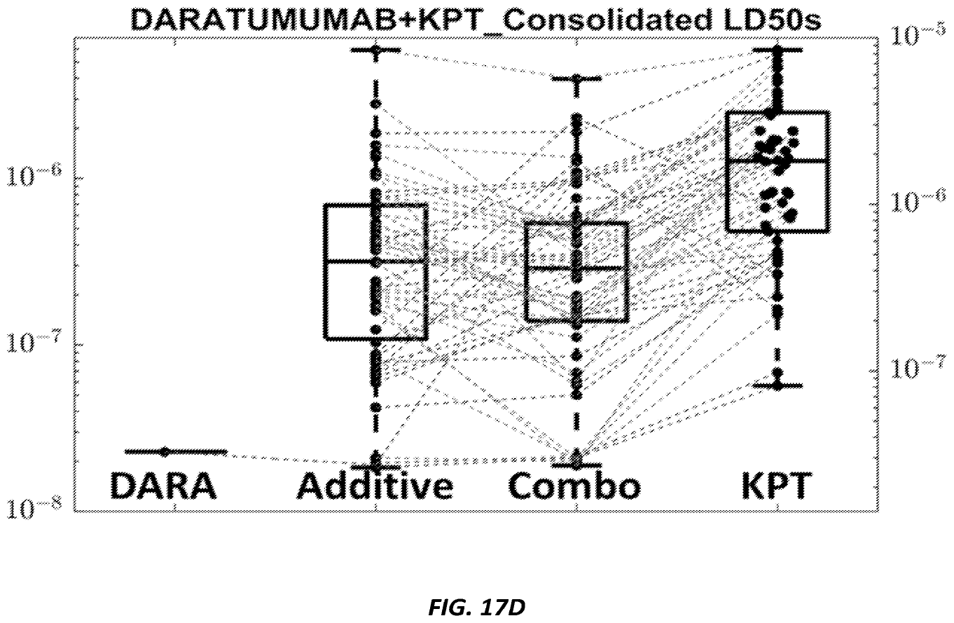

FIGS. 17A to 17D show box and whisker plots to show the extent of ex vivo synergy for Selinexor and dexamethasone (FIG. 17A) Selinexor and pomalidomide (FIG. 17B), Selinexor and elotuzumab (FIG. 17C), and Selinexor and daratumumab (FIG. 17D), where the first and fourth columns show LD₅₀s for the two single agents, the second column shows the LD₅₀ for the additive response, and the third column shows the LD₅₀s for the combination. Each line represents a patient, red indicates synergy and blue indicates antagonism.

We inspected the transcriptomic map for Selinexor and dexamethasone and notice that clusters highlighted for intensity of synergy are also highlighted for intensity of resistance to Selinexor alone; implying that the combination benefits Selinexor resistant patients more. The biomarkers listed give an insight into the mechanisms that might be involved in this process.

In Selinexor and elotuzumab, the highlighted gene cluster is enriched for intensity of antagonism for the combination and for resistance in Selinexor alone. This is also evident from the whisker box plot, where we see more blue lines at the top.

Selinexor and pomalidomide, on the other hand, has a cluster that's enriched for intensity of antagonism for the combination but for sensitivity in Selinexor alone. This is also evident from the whisker box plot, with more blue lines at the bottom.

Finally, the combinations Selinexor with Daratumumab and Selinexor with Dexamethasone have several clusters that are enriched for complimentary conditions as highlighted. This may imply that patients who show antagonism to Selinexor and dexamethasone, may show synergy via the combination Selinexor and daratumumab.

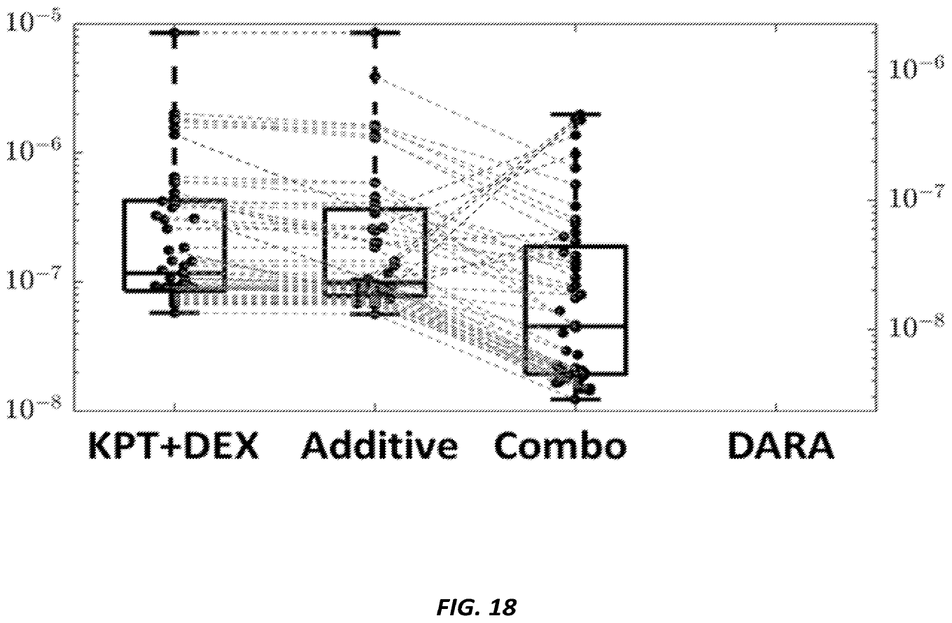

This is validated in the whisker box (FIG. 18), where adding Daratumumab to the combination of Selinexor and Dexamethasone significantly lowered the LDsos even though the pair Selinexor and daratumumab doesn't show synergy.

### D. References

Ayesha, S., Hanif, M. K. & Talib, R. Overview and comparative study of dimensionality reduction techniques for high dimensional data. Information Fusion 59, 44-58.
Bezdek, J. C., Ehrlich, R. & Full, W. FCM: The fuzzy c-means clustering algorithm. Computers & Geosciences 10, 191-203.
Hornbeck, P. V. Et al. Phosphositeplus: a comprehensive resource for investigating the structure and function of experimentally determined post-translational modifications in man and mouse. Nucleic Acids Research 40, D261-D270, doi: 10.1093/nar/gkr1122 (2011).
Kumar, S. K. Et al. Multiple myeloma. Nature Reviews Disease Primers 3, 17046, doi: 10.1038/nrdp.2017.46 (2017).
Lachmann, A. Et al. Chea: transcription factor regulation inferred from integrating genome-wide chip-X experiments. Bioinformatics 26, 2438-2444, doi: 10.1093/bioinformatics/btq466 (2010).
Moore, J. E. Et al. Expanded encyclopaedias of DNA elements in the human and mouse genomes. Nature 583, 699-710, doi: 10.1038/s41586-020-2493-4 (2020).
Silva, A. Et al. An Ex Vivo Platform for the Prediction of Clinical Response in Multiple Myeloma. Cancer Research 77, 3336-3351, doi: 10.1158/0008-5472.Can-17-0502 (2017).
Silva, A., Jacobson, T., Meads, M., Distler, A. & Shain, K. An Organotypic High Throughput System for Characterization of Drug Sensitivity of Primary Multiple Myeloma Cells. Jove-J Vis Exp, doi:ARTN e5307010.3791/53070 (2015).
Subramanian, A. Et al. Gene set enrichment analysis: A knowledge-based approach for interpreting genome-wide expression profiles. Proceedings of the National Academy of Sciences 102, 15545-15550, doi: 10.1073/pnas.0506580102 (2005).
Sudalagunta, P. Et al. A pharmacodynamic model of clinical synergy in multiple myeloma. Ebiomedicine 54, 102716, doi: 10.1016/j.ebiom.2020.102716 (2020).
Teschendorff, A. E. & Feinberg, A. P. Statistical mechanics meets single-cell biology. Nat Rev Genet, doi: 10.1038/s41576-021-00341-z (2021).
Van der Maaten, L. & Hinton, G. Visualizing Data using t-SNE. J Mach Learn Res 9, 2579-2605 (2008).
Yadav, B. Et al. Quantitative scoring of differential drug sensitivity for individually optimized anticancer therapies. Sci Rep-Uk 4, doi:ARTN 519310.1038/srep05193 (2014).
Yang, C.-Y. Et al. Phosphopoint: a comprehensive human kinase interactome and phospho-protein database. Bioinformatics 24, i14-i20, doi: 10.1093/bioinformatics/btn297 (2008).

### FIG. 20 References

1 Dibb, M. et al. The FOXM1-PLK1 axis is commonly upregulated in oesophageal adenocarcinoma. British Journal of Cancer 107, 1766-1775, doi: 10.1038/bjc.2012.424 (2012).
2 Fu, Z. et al. Plk1-dependent phosphorylation of FoxM1 regulates a transcriptional programme required for mitotic progression. Nature Cell Biology 10, 1076-1082, doi: 10.1038/ncb1767 (2008).
3 Yu, W. et al. Silencing forkhead box MI promotes apoptosis and autophagy through SIRT7/mTOR/IGF2 pathway in gastric cancer cells. Journal of Cellular Biochemistry 119, 9090-9098, doi:https://doi.org/10.1002/jcb.27168 (2018). al. FOXM1 contributes to docetaxel resistance in castration-resistant prostate cancer by inducing AMPK/mTOR-mediated
4 Lin, J.-Z. et autophagy. Cancer Lett 469, 481-489, doi:10.1016/j.canlet.2019.11.014 (2020).
5 Lee, N.-R. et al. Inactivation of the Akt/FOXM1 Signaling Pathway by Panobinostat Suppresses the Proliferation and Metastasis of Gastric Cancer Cells. International Journal of Molecular Sciences 22, 5955 (2021).
6 Zhang, N. et al. FoxM1 Promotes β-Catenin Nuclear Localization and Controls Wnt Target-Gene Expression and Glioma Tumorigenesis. Cancer Cell 20, 427-442, doi:https://doi.org/10.1016/j.ccr.2011.08.016 (2011).
7 Xu, F. et al. Anthelmintic pyrvinium pamoate blocks Wnt/β-catenin and induces apoptosis in multiple myeloma cells. Oncology letters 15, 5871-5878, doi: 10.3892/ol.2018.8006 (2018).
8 Gomes, A. R., Zhao, F. & Lam, E. W. F. Role and regulation of the forkhead transcription factors FOXO3a and FOXM1 in carcinogenesis and drug resistance. Chin J Cancer 32, 365-370, doi:10.5732/cjc.012.10277 (2013).
9 Zhang, C. et al. Gli1 promotes colorectal cancer metastasis in a Foxm1-dependent manner by activating EMT and PI3K-AKT signaling. Oncotarget 7 (2016).
10 Halasi, M. & Gartel, A. L. Suppression of FOXM1 Sensitizes Human Cancer Cells to Cell Death Induced by DNA-Damage. PLOS ONE 7, e31761, doi:10.1371/journal.pone.0031761 (2012).
11 Chesnokov, M. S. et al. FOXM1-AKT Positive Regulation Loop Provides Venetoclax Resistance in AML. Frontiers in Oncology 11, doi: 10.3389/fonc.2021.696532 (2021). 20
12 Webb, B. M. et al. TGF-&#x3b2;/activin signaling promotes CDK7 inhibitor resistance in triple-negative breast cancer cells through upregulation of multidrug transporters. Journal of Biological Chemistry 297, doi: 10.1016/j.jbc.2021.101162 (2021).
13 Song, S. et al. Foxm1 is a critical driver of TGF-β-induced EndMT in endothelial cells through Smad2/3 and binds to the Snail promoter. Journal of Cellular Physiology 234, 9052-9064, doi:https://doi.org/10.1002/jcp.27583 (2019).
14 Markovina, S. et al. Bortezomib-Resistant Nuclear Factor-κB Activity in Multiple Myeloma Cells. Molecular Cancer Research 6, 1356-1364, doi: 10.1158/1541-7786.Mcr-08-0108 (2008).
15 Duvefelt, C. F. et al. Increased resistance to proteasome inhibitors in multiple myeloma mediated by cIAP2—implications for a combinatorial treatment. Oncotarget 6 (2015).
16 Cai, Y. et al. Crm1-Dependent Nuclear Export of Bach1 is Involved in the Protective Effect of Hyperoside on Oxidative Damage in Hepatocytes and CCl4-induced Acute Liver Injury. J Inflamm Res 14, 551-565, doi: 10.2147/JIR.S279249 (2021).
17 Igarashi, K., Nishizawa, H., Saiki, Y. & Matsumoto, M. The transcription factor BACH1 at the crossroads of cancer biology: From epithelial-mesenchymal transition to ferroptosis. Journal of Biological Chemistry 297, 101032, doi:https://doi.org/10.1016/j.jbc.2021.101032 (2021).

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method of measuring tumor chemosensitivity in a subject with multiple myeloma comprising obtaining multiple myeloma cells from a subject; culturing said multiple myeloma cells; contacting the multiple myeloma cells with one or more individual anti-cancer agents and/or combinations of two or more anti-cancer agents; taking an image of said multiple myeloma cells at least two times; and applying an image analysis algorithm to said images to determine viability across time and/or concentration thereby forming a model of drug sensitivity.

2. The method of claim 1, wherein the multiple myeloma cells are cultured in the presence of stroma, collagen, and/or plasma.

3. The method of any of claim 1 or 2, wherein at least one image is obtained prior to the contact with the anti-cancer agent.

4. The method of any of claims 1-3, wherein the multiple myeloma cells are imaged for at least 2, 3, 4, 5, 6, 7, 8, 9, 10 11, 12, 13, 14, 15, 16, 17, 18, 19, 20, 21, 22, 23, 24, 25, 26, 27, 28, 29, 30, or 31 days.

5. The method of any of claims 1-4, wherein an image is made of the multiple myeloma cells every 5, 10, 15, 20, 25, 30, 35, 40, 45, 50, 55, 60, 65, 70, 75, 80, 85, 90, 95, 100, 105, 110, 115, 120 min, 3, 4, 5, 6, 7, 8, 9, 10, 11, 12, 13, 14, 15, 16, 17, 18, 19, 20, 21, 22, 23, 24, 36, 48, 60, 72 hours.

6. The method of any of claims 1-5, wherein the multiple myeloma cells are separately contacted with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more individual anti-cancer agents.

7. The method of any of claims 1-6, wherein the multiple myeloma cells are separately contacted with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, or more combinations of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 individual anti-cancer agents.

8. The method of any of claims 1-7, wherein the image is a bright field image.

9. The method of claim 8, wherein viability of cells is determined by assessing non-translational cellular membrane motion of the cell after stage drift and field vibrations are excluded.

10. The method of any of claims 1-9, further comprising coupling drug sensitivity model to clinical trials to establish a patient's response to single agents and combinations thereby establishing a patient's early objective response (EOR) to each of the drugs tested and quantify synergistic effects in combinations.

11. The method of any of claims 1-10, further comprising applying or adjusting a patient's treatment regimen based on the sensitivities.

12. A method of identifying gene signatures associated with anti-cancer therapy resistance for multiple myeloma comprising obtaining multiple myeloma cells from a subject; culturing said multiple myeloma cells; contacting the multiple myeloma cells with one or more individual anti-cancer agents and/or combinations of two or more anti-cancer agents; taking an image of said multiple myeloma cells at least two times; applying an image analysis algorithm to said imagens to determine viability across time and/or concentration thereby forming a model of drug sensitivity; in parallel to assaying cellular sensitivity to one or more anti-cancer agents, sequencing multiple myeloma cells obtained from the patient; analyzing the gene expression profiled obtained from the sequence information in combination with the drug sensitivity data to determine gene signatures associated with therapy resistance.

13. The method of claim 12, further comprising applying gene signature data to a gene regulatory network (GRN) model to identify transcriptional regulatory mechanisms driving therapy resistance.

14. The method of claim 12 or 13, further comprising repeating the analysis from two or more patients to identify gene signatures and/or transcriptional regulatory mechanisms driving therapy resistance common to a cohort.

15. A method of identifying therapeutic regimens for a subject comprising identifying gene signatures using the method of claim 12 and identifying transcriptional regulatory mechanisms driving therapy resistance using the method of claim 13; applying gene signature and GRN model information to identify novel therapeutic strategies either as a combination, or sequential therapy.

16. A method of identifying novel therapeutic regimens for the treatment of multiple myeloma comprising identifying gene signatures using the method of claim 12 from two or more patients and identifying transcriptional regulatory mechanisms driving therapy resistance using the method of claim 13 from two or more patients; applying gene signature and GRN model information to identify novel therapeutic strategies either as a combination, or sequential therapy.

17. A computer-implemented method, comprising:
receiving patient data for a plurality of patients having a disease, the patient data comprising respective RNA sequencing data and respective ex vivo drug response data for the plurality of patients; and
identifying one or more gene signatures for therapy resistance.

18. The computer-implemented method of claim 17, wherein the step of identifying one or more gene signatures for therapy resistance comprises performing a cluster analysis.

19. The computer-implemented method of claim 17 or 18, wherein the plurality of patients represent a heterogenous cohort of patients having early-, middle-, and late-stages of the disease.

20. The computer-implemented method of any one of claims 17-19, wherein the disease is multiple myeloma.

21. The computer-implemented method of any one of claims 17-20, further comprising training a machine learning model with a dataset created from the patient data and the identified one or more gene signatures for therapy resistance, wherein the machine learning model is configured to predict drug response.

22. The computer-implemented method of claim 21, further comprising inputting, into the trained machine learning model, RNA sequencing data for a specific patient; and predicting, using the trained machine learning model, the specific patient's response to a drug.

23. The computer-implemented method of any one of claims 17-22, further comprising creating a gene regulatory network model with a dataset created from the patient data and the identified one or more gene signatures for therapy resistance, wherein the gene regulatory network model is configured to provide therapeutic strategies.

24. The computer-implemented method of claim 23, further comprising inputting, into the gene regulatory network model, RNA sequencing data for a specific patient; and providing, using the gene regulatory network model, a therapeutic strategy for the specific patient.

25. The computer-implemented method of claim 24, wherein the therapeutic strategy is a combination or sequential therapy.

26. A method comprising:
providing a trained machine learning model, the trained machine learning model being configured to predict drug response;
inputting, into the trained machine learning model, RNA sequencing data for a specific patient; and
predicting, using the trained machine learning model, the specific patient's response to a drug.

27. The method of claim 26, further comprising administering the drug to the specific patient.

28. A method comprising:
providing a gene regulatory network model, wherein the gene regulatory network model is configured to provide therapeutic strategies;
inputting, into the gene regulatory network model, RNA sequencing data for a specific patient; and
predicting, using the gene regulatory network model, a therapeutic strategy for the specific patient.

29. The method of claim 28, further comprising administering the therapeutic strategy to the specific patient.

30. A method comprising:
receiving patient data for a plurality of patients having a disease, the patient data comprising respective RNA sequencing data and respective ex vivo drug response data for the plurality of patients;
receiving RNA sequencing data for a specific patient;
deploying a trained machine learning model in response to the RNA sequencing data for the specific patient, wherein the trained machine learning model is configured to predict drug response;
deploying a gene regulatory network model in response to the RNA sequencing data for the specific patient, wherein the gene regulatory network model is configured to provide therapeutic strategies;
simulating, using a network controllability approach, respective effects of a plurality of targeted therapies on the specific patient using the patient data, an output of the trained machine learning model, and an output of the gene regulatory network model; and
predicting a drug response for the specific patient based on the simulation.

31. The method of claim 30, further comprising administering the drug to the specific patient.
